(19) United States
(12) Patent Application Publication
(10) Pub. No.: US 2011/0053773 A1
(43) Pub. Date: Mar. 3, 2011

(54) METHODS OF IMPROVING NUTRITIONAL VALUE OF PLANTS

(75) Inventors: GREGORY RUSSELL ARMEL, Knoxville, TN (US); Dean Adam Kopsell, Knoxville, TN (US); James T. Brosnan, Knoxville, TN (US); Brandon J. Horvath, Knoxville, TN (US); John C. Sorochan, Knoxville, TN (US)

(73) Assignee: UNIVERSITY OF TENNESSEE RESEARCH FOUNDATION, KNOXVILLE, TN (US)

(21) Appl. No.: 12/875,328

(22) Filed: Sep. 3, 2010

Related U.S. Application Data
(60) Provisional application No. 61/239,602, filed on Sep. 3, 2009.
(60) Provisional application No. 61/262,580, filed on Nov. 19, 2009.
(60) Provisional application No. 61/356,197, filed on Jun. 18, 2010.

(51) Int. Cl. A01N 25/32; C12Q 1/02; A01N 57/16; A01N 43/40; A01N 43/88; A01N 43/70; A01N 43/653; A01N 47/40; A01N 37/22; A01N 35/06; A01P 3/00; A01P 21/00; A01P 7/04; A01P 5/00; A01P 13/00; A01P 1/00

(52) U.S. Cl. 504/107; 435/29; 504/103; 504/108; 504/128; 504/130; 504/131; 504/133; 504/134; 504/139; 504/141; 504/149; 504/234; 504/348

(57) ABSTRACT

The subject application provides methods for the direct or indirect improvement of levels of key phytonutrients and/or stress tolerance in plants. Methods of providing for the improvement in key phytonutrient levels and/or stress tolerance in plants are provided through the application of safeners, herbicides, fungicides, insecticides, nematicides, miticides, defoliants/desiccants, antibiotics, and/or plant growth regulators to plants. Agricultural products arising from the disclosed methods are also provided.

### CROSS-REFERENCE TO RELATED APPLICATIONS

This application claims the benefit of U.S. Provisional Application Ser. No. 61/239,602, filed Sep. 3, 2009; Ser. No. 61/262,580, tiled Nov. 19, 2009; and Ser. No. 61/356,197, filed Jun. 18, 2010, the disclosures of which are hereby incorporated by reference in their entirety, including all figures, tables and amino acid or nucleic acid sequences.

### BACKGROUND OF THE INVENTION

Human and other animals are dependent on key phytonutrients produced or found in plants for optimal health. These phytonutrients include but are not limited to lipids (i.e., oils, fatty acids, saturated fatty acids, non-saturated fatty acids, steroids, other), vitamins [Vitamin A (retinol), Vitamin B₁ (thiamine), Vitamin B₂ (riboflavin), Vitamin B₃ (niacin), Vitamin B₁₂, Folic acid (folacin), Vitamin C (Ascorbic acid), Vitamin D, Vitamin E (tocopherols), Vitamin K, other], minerals (i.e. N, P, K, Ca, Mg, S, Cl, Co, Cu, Fe, Mn, Mo, Na, Ni, Zn, etc.), proteins, amino acids (i.e., histidine, valine, leucine, isoleucine, lysine, methionine, cysteine, phenylalanine, tyrosine, threonine, tryptophan, etc.), carbohydrates [including but not limited to starch, fiber, cellulose, and sugars (i.e., sucrose, fructose, glucose, maltose, raffanose, etc.)] carotenoid/xanthophyll antioxidants (i.e., beta-carotene, lycopene, lutein, zeaxanthin, antheraxanthin, etc.), glucosinolates (i.e., glucobrassicin, sinalbin, etc.) and phenolic compounds (i.e. capsaicin, eugenol, polyphenols, salicylic acid, anthocyanins, tannins, resveratrol, etc.). Current methods used to improve the quantity and/or quality of these types of plant nutrients include adjusting crop fertility, molecular genetic manipulation, plant breeding, and variations in processing and handling of crops.

Moreover, changes in the level of phytonutrients in a variety of crops can provide many meaningful benefits including improved nutrition and flavor enhancement of crops for humans, livestock, wildlife, and/or pets. In addition, processed foods (i.e., cereals, pasta, soups, etc.) or other edible substance derived from crops (i.e., processed sugar, high fructose corn syrup, guacamole, maple syrup, flavor enhancers, spices, etc.) can also have their nutritional composition enhanced by changes in the levels of phytonutrients contained in their crop of origin. In addition, changes in levels of phytonutrients may also help facilitate more efficient fermentation for crops (i.e., barley, rice, corn, hops, wheat, rye, grapes, agave, potato, sugarcane, switchgrass, etc.) used in the manufacturing of alcohol for consumption (such as wines, beer, and/or distilled spirits), biofuels (i.e., ethanol, butanol, etc.), and other industrial and consumer uses (i.e., antiseptics, etc.). Also, changes in levels of phytonutrients can improve the harvestability, fiber quality, processing efficiency, the rate of ripening, and/or shelf-life of key crops. In addition, changing the levels of phytonutrients can enhance the production of or improve the quality of other plant derived products including but not limited to pharmaceuticals, dietary supplements, biologically derived materials (i.e., cotton fiber, forest products, lumber, paper, biodegradable plastics, hemp rope, etc.), or biocatalysts.

Herbicides are chemicals that kill plants by inhibiting or modifying key biochemical processes. Selective herbicides control weeds safely in crops because they are exuded, metabolized, compartmentalized, or not absorbed or transported to the target site in tolerant crops in comparison to sensitive weed species. Fungicides include chemicals that kill fungi or inhibit the growth, reproduction and/or infestation of fungi. Plant growth regulators, also known as plant hormones, include natural or synthetic chemicals that regulate or alter the growth of a plant or plant part. Insecticides include chemicals that kill or inhibit the growth, reproduction, and/or infestation of insects. Nematicides include chemicals that kill or inhibit the growth, reproduction, and/or infestation of nematodes. Miticides include chemicals that kill or inhibit the growth, reproduction, and/or infestation of mites. Defoliants/desiccants include chemicals that are applied to aid in plant drying, leaf kill, leaf drop, or aid in other aspects of crop harvest or vegetation management. Antibiotics include chemicals that are applied to aid in the control of bacteria and other plant diseases. Herbicides, fungicides, insecticides, nematicides, miticides, defoliants/desiccants, antibiotics, and plant growth regulators may be collectively categorized with other chemicals and referred to as pesticides and/or agrochemicals.

Crops may be naturally tolerant to a specific herbicide, fungicide, insecticide, nematicides, miticides, defoliants/desiccants, antibiotics, or plant growth regulator, or may become selective to one or more of these agrochemicals through molecular genetic manipulation, chemically induced gene mutation techniques, or through plant breeding. However, herbicides, fungicides, insecticides, nematicides, miticides, defoliants/desiccants, antibiotics, and plant growth regulators that are considered selective to a crop may still cause some injury in these crops due to variations in application timing, application rate, tank mixtures with other agrochemicals, climate, soil type, formulations, adjuvants, or crop varietal sensitivity. This injury creates stress responses in plants that may not be obvious to those skilled in the art of this invention.

### DETAILED DISCLOSURE OF THE INVENTION

It has now been discovered that plant stress responses to the applications of safeners, herbicides, fungicides, insecticides, nematicides, miticides, defoliants/desiccants, antibiotics, and/or plant growth regulators leads to direct or in-direct improvements in the levels of key phytonutrients and/or stress tolerance in plants. Stress, as used herein, describes, collectively, any condition or conditions that can have negative impacts on plant quality and/or overall performance. Stress responses appear as continuums from very rapid physiological changes to much slower morphological changes. Antioxidant compounds within plants, including for example, plant secondary metabolites, such as terpenes (carotenoids and essential oils), phenolics (flavonoids and anthocyanins), and nitrogen-containing compounds (alkaloids and glucosinolates), serve functional roles to overcome the negative consequences to plant growth and development caused by stress. As a result, their production may increase or decrease in response to various forms of abiotic environmental stress, such as drought, elevated temperatures, and nutrient imbalances. Applications of safeners, herbicides, fungicides, insecticides, nematicides, miticides, defoliants/desiccants, antibiotics, and/or plant growth regulators may enhance antioxidant metabolic pathways in plants, including for example, quenching the formation of reactive oxygen, chlorophyll, and other detrimental species promoted under abiotic stress conditions. Therefore, safeners, herbicides, fungicides, insecticides, nematicides, miticides, defoliants/desiccants, antibiotics, and/or plant growth regulators, applied either alone or in combination with diluents, adjuvants, fertilizers, nutrient based solutions, other agrochemicals, crop safeners and/or other additives, are useful for improving the levels of key phytonutrients, enhancing stress tolerance, and thus, producing more economically valuable and healthy plants.

Overall this invention applies to any chemical designated as a pro-herbicide, herbicide, pro-fungicide, fungicide, pro-insecticide, insecticides, pre-nematicide, nematicide, pro-miticide, miticide, pro-defoliant, defoliant, pro-antibiotic, antibiotic, pro-plant growth regulator, or plant growth regulator recognized as having any herbicidal, fungicidal, insecticidal, nematicidal, miticidal, anti-bacterial, anti-viral, or plant growth regulating properties. In certain aspects of the invention a herbicide, fungicide, insecticide, nematicides, miticide, defoliant, antibiotic, and/or plant growth regulators can be applied at a rate of 0.006 g ai/ha to approximately 6,000 g ai/ha, more preferred at a rate of 0.03 g ai/ha to 3,000 g ai/ha, and most preferred at a rate of 0.5 to 1,500 g ai/ha in a single or multiple applications, alone or in mixtures with various ratios of one or more of the following 1) diluents used to create a formulated product (i.e., solid and/or liquid which include but are not limited to water, glycerine, propylene glycols, paraffins, acetates, other) 2) herbicides (i.e., naturally derived or synthetic, in formulated or technical form); 3) fungicides (i.e., naturally derived or synthetic, in formulated or technical form); 4) insecticides (i.e., naturally derived or synthetic, in formulated or technical form); 5) nematicides (i.e., naturally derived or synthetic, in formulated or technical form); 6) miticides (i. e. , naturally derived or synthetic, in formulated or technical form); 7) defoliants/desiccants (i.e., naturally derived or synthetic, in formulated or technical form); 8) antibiotics (i.e., naturally derived or synthetic, in formulated or technical form); 9) plant growth regulators (i.e., naturally derived or synthetic, in formulated or technical form); 10) adjuvants (i.e., non-ionic surfactants, petroleum or seed based oils or organosilicones); 11) fertilizers and other nutrient based solutions (including but not limited to products offering containing individual nutrients or mixtures of multiple macronutrients such as nitrogen, potassium, and phosphorous and/or micronutrients such as manganese, boron, and zinc); 12) other agrochemicals (including but not limited to herbicides, insecticides, fungicides, antibiotics, nematicides, miticides, defoliants/desiccants, antibiotics, plant growth regulators, etc.); 13) other additives (i.e., anti-foaming agents, dyes, etc.); 14) crop safeners (i.e., benoxacor, cloquintocet, cyometrinil, dichlormid, dicyclonon, dietholate, fenchlorazole, fenclorim, flurazole, fluxofenim, furilazole, isoxadifen, mefenpyr, mephenate, naphthalic anhydride, oxabetrinil, cyprosulfamide, or others) with the intent of stressing or enhancing plant biochemical processes in order to increase key phytonutrients and/or enhance plant stress tolerance. Mixtures of one or more herbicides, fungicides, insecticides, nematicides, miticides, defoliants/desiccants, antibiotics, and/or plant growth regulators with diluents, adjuvants, fertilizers, nutrient based solutions, other agrochemicals, crop safeners and/or other additives would expect additive or synergistic improvements concurrently in control of pests (i.e., weeds, insects, nematodes, mites, fungal/viral/bacterial pathogens, etc.), plant growth and appearance, levels of one or more phytonutrients, plant stress tolerance, and crop yield.

Crops that would benefit from this invention include but are not limited to alfalfa, barley, corn (maize), popcorn, sweet corn, sorghum, cotton, soybean, sugarbects, sunflower, sugarcane, rape, canola, peanuts, rice, oats, triticale, rye, agave, wheat, potato, tomato, fruits (i.e., apple, apricot, avocado, breadfruit, banana, blackberry, blackcurrant, blueberry, cherimoya, cherry, clementine, coconut, cranberry, durian, fig, grapefruit, grape, guava, jackfruit, kiwi, lemon, lime, loganberry, mandarin, mango, mangosteen, melon, nectarine, orange, papaya, peach, pear, persimmon, pineapple, plum, pomegranate, quince, satsuma, strawberry, tamarillo, ugli fruit, watermelon etc.), nuts (i.e., almond, beech, butternut, brazil nut, candlenut, cashew, chestnuts, colocynth, filbert, hickory, pecan, shagbark hickory, kola nut, macadamia, mamoncillo, maya nut, oak acorns, ogbono nut, paradise nut, pili nut, pistachio, walnut, etc.), vegetables (i.e., asparagus, artichoke, leafy greens, melons, snapbean, lima bean, cabbage, pea, spinach, pumpkin, onions, garlic, squash, eggplant, carrots, broccoli, sweet potato, zucchini, etc.), turfgrass (i.e., bahiagrass, bluegrass, buffalograss, fescue, bentgrass, bermudagrass, ryegrass, St. Augustinegrass, zoysiagrass, etc.), forage grasses, switchgrass, forage legumes (i.e., clover, lesepedeza, etc.), ornamental plants, forest plants (i.e., hardwood trees, pines, shrubs, vines, or wild flowers), herbs or other flavor enhancing crops (i.e., peppermint, spearmint, thyme, basil, coriander, dill, rosemary, Irish moss, arrowroot, sesame, etc.), and plantation crops (i.e., oil palm, cocoa, coffee, hops, pineapple, eucalyptus, etc.). Crops that may be treated as disclosed herein may also be genetically modified crops, a cultivar produced through common methods of plant breeding (i.e., asexual propagation, chemical/radioactive induced mutations, pedigree breeding, backcrossing, etc.), or crops that have not been genetically modified or produced through common plant breeding techniques.

Accordingly, one aspect of the invention provides agricultural products, produced by the crop plants mentioned above, having enhanced nutritional value and/or stress tolerance. These agricultural products may be produced by crop plants that are genetically modified or that have not been genetically modified. These agricultural products are produced using the methods disclosed herein and contain enhanced or increased levels (amounts) of lipids (i.e., oils, fatty acids, saturated fatty acids, non-saturated fatty acids, steroids, other), vitamins (Vitamin A (retinol), Vitamin B₁ (thiamine), Vitamin B₂ (riboflavin), Vitamin B₃ (niacin), Vitamin B₁₂, Folic acid (folacin), Vitamin C (Ascorbic acid), Vitamin D, Vitamin E (tocopherols), Vitamin K, or other vitamins), minerals (i.e. calcium, iron, iodide, fluoride, zinc, etc.), proteins, amino acids (i.e., histidine, valine, leucine, isoleucine, lysine, methionine, cysteine, phenylalanine, tyrosine, threonine, tryptophan, etc.), carbohydrates (including but not limited to starch, fiber, cellulose, and sugars (i.e., sucrose, fructose, glucose, maltose, raffanose, etc.) carotenoid/xanthophyll antioxidants (i.e., beta-carotene, lycopene, lutein, zeaxanthin, antheraxanthin, etc.), glucosinolates (i.e., glucobrassicin, sinalbin, etc.) and phenolic compounds (i.e. capsaicin, eugenol, polyphenols, salicylic acid, anthocyanins, tannins, resveratrol, etc.), and minerals (i.e. N, P, K, Ca, Mg, S, Cl, Co, Cu, Fe, Mn, Mo, Na, Ni, Zn, etc.).

Another aspect of this invention involves applications of one or more safeners, herbicides, fungicides, insecticides, nematicides, miticides, defoliants/desiccants, antibiotics, and/or plant growth regulators, either alone or in combinations with each other and with diluents, adjuvants, fertilizers, nutrient based solutions, other agrochemicals, additional crop safeners and/or other additives, in order to enhance plant nutritional value and/or stress tolerance.

In an embodiment, applications of herbicides, fungicides, insecticides, nematicides, miticides, defoliants/desiccants, antibiotics, and/or plant growth regulators (PGRs), safeners either alone or in combination with diluents, adjuvants, fertilizers, nutrient based solutions, other agrochemicals, additional crop safeners and/or other additives enhance, directly or indirectly, improve the production of lipids (i.e., oils, fatty acids, steroids, other), vitamins (Vitamin A (retinol), Vitamin B₁ (thiamine), Vitamin B₂ (riboflavin), Vitamin B₃ (niacin), Vitamin B₁₂, Folic acid (folacin), Vitamin C (Ascorbic acid), Vitamin D, Vitamin E (tocopherols), Vitamin K, or other vitamins), minerals (i.e. calcium, iron, iodide, fluoride, zinc, etc.), proteins, amino acids (i.e., histidine, valine, leucine, isoleucine, lysine, methionine, cysteine, phenylalanine, tyrosine, threonine, tryptophan, etc.), carbohydrates (including but not limited to starch, fiber, cellulose, and sugars (i.e., sucrose, fructose, glucose, maltose, raffanose, etc.)) carotenoid/xanthophyll antioxidants (i.e., beta-carotene, lycopene, lutein, zeaxanthin, antheraxanthin, etc.), glucosinolates (i.e., glucobrassicin, sinalbin, etc.), phenolic compounds (i.e. capsaicin, eugenol, polyphenols, salicylic acid, anthocyanins, tannins, resveratrol, etc.), and minerals (i.e. N, P, K, Ca, Mg, S, Cl, Co, Cu, Fe, Mn, Mo, Na, Ni, Zn, etc.).

In another embodiment, applications of herbicides, fungicides, insecticides, nematicides, miticides, defoliants/desiccants, antibiotics, safeners and/or plant growth regulators, particularly at elevated, registered, or reduced application rates to enhance plant stress tolerance in response to a variety of abiotic conditions, such as for example, drought, elevated temperatures, nutrient imbalances, and applications of agrochemicals such as insecticides, fungicides, herbicides, nematicides, miticides, defoliants/desiccants, antiobiotics, plant growth regulators, others. In order to optimally enhance stress tolerance, application rates of herbicides, fungicides, insecticides, nematicides, miticides, defoliants/desiccants, antibiotics, and/or plant growth regulators can be applied at a rate of 0.006 g ai/ha to approximately 6,000 g ai/ha, more preferred at a rate of 0.03 g ai/ha to 3,000 g ai/ha, and most preferred at a rate of 0.5 to 1,500 g ai/ha in a single or multiple applications, alone or in mixtures with various ratios of one or more of the following 1) diluents used to create a formulated product (i.e., solid and/or liquid which include but are not limited to water, glycerine, propylene glycols, paraffins, acetates, other) 2) herbicides (i.e., naturally derived or synthetic, in formulated or technical form); 3) fungicides (i.e., naturally derived or synthetic, in formulated or technical form); 4) insecticides (i.e., naturally derived or synthetic, in formulated or technical form); 5) nematicides (i.e., naturally derived or synthetic, in formulated or technical form); 6) miticides (i.e., naturally derived or synthetic, in formulated or technical form); 7) defoliants/desiccants (i.e., naturally derived or synthetic, in formulated or technical form); 8) antibiotics (i.e., naturally derived or synthetic, in formulated or technical form); 9) plant growth regulators (i.e., naturally derived or synthetic, in formulated or technical form); 10) adjuvants (i.e., non-ionic surfactants, petroleum or seed based oils or organosilicones); 11) fertilizers and other nutrient based solutions (including but not limited to products offering containing individual nutrients or mixtures of multiple macronutrients such as nitrogen, potassium, and phosphorous and/or micronutrients such as manganese, boron, and zinc); 12) other agrochemicals (including but not limited to herbicides, insecticides, fungicides, antibiotics, nematicides, miticides, defoliants/desiccants, antibiotics, plant growth regulators, etc.); 13) other additives (i.e., anti-foaming agents, dyes, etc.); 14) crop safeners (i.e., benoxacor, cloquintocet, cyometrinil, dichlormid, dicyclonon, dietholate, fenchlorazole, fenclorim, flurazole, fluxofenim, furilazole, isoxadifen, mefenpyr, mephenate, naphthalic anhydride, oxabetrinil, cyprosulfamide, other) with the intent of stressing or enhancing plant biochemical processes in order to increase key phytonutrients and/or enhance plant stress tolerance. Mixtures of one or more herbicides, fungicides, insecticides, nematicides, miticides, defoliants/desiccants, antibiotics, and/or plant growth regulators with diluents, adjuvants, fertilizers, nutrient based solutions, other agrochemicals, crop safeners and/or other additives would expect additive or synergistic improvements concurrently in control of pests (i.e., weeds, insects, nematodes, mites, fungal/viral/bacterial pathogens, etc.), plant growth and appearance, levels of one or more phytonutrients, plant stress tolerance, and crop yield.

In a specific embodiment, applications of safeners, herbicides, fungicides, insecticides, nematicides, miticides, defoliants/desiccants, antibiotics, and/or plant growth regulators stimulate the uptake and/or modify the partitioning of essential minerals (such as N, P, K, Ca, Mg, S, Cl, Co, Cu, Fe, Mn, Mo, Na, Ni, Zn, etc.), and ultimately lead to improved stress tolerance through changes in plant osmoticum concentrations.

In another specific embodiment, applications of safeners, herbicides, fungicides, insecticides, nematicides, miticides, defoliants/desiccants, antibiotics, and/or plant growth regulators improve the production one or more anti-oxidant compound, such as terpenes (carotenoids and essential oils), phenolics (flavonoids and anthocyanins), and nitrogen-containing compounds (alkaloids and glucosinolates) for improved stress tolerance in plants.

In another specific embodiment, application of safeners, herbicides fungicides, insecticides, nematicides, miticides, defoliants/desiccants, antibiotics, and/or plant regulators can improve the production of or partitioning of certain lipids (i.e., fatty acids, steroids, etc.) proteins, amino acids, vitamins (i.e, Vitamin E, Vitamin A, etc.), and/or carbohydrates (i.e., sugars, fiber, cellulose, etc.) for improved stress tolerance.

In certain embodiments, applications of safeners, herbicides, fungicides, insecticides, nematicides, miticides, defoliants/desiccants, antibiotics, and/or plant growth regulators result in an increase in levels of one or more phytonutrients by approximately 5%, 10%, 15%, 20%, 25%, 30%, 40%, 50%, 60%, 70%, 80%, 90%, 100%, or greater than 100%.

The method disclosed herein can utilize any safener. Non-limiting examples of acceptable safeners include: benoxacor, cloquintocet, cyometrinil, cyprosulfamide, dichlormid, dicyclonon, dietholate, fenchlorazole, fenclorim, flurazole, fluxofenim, furilazole, isoxadifen, jiecaowan, jiecaoxi, mefenpyr, mephenate, naphthalic anhydride, oxabetrinil, daimuron, mefenpyr-diethy, cloquintocet-mexyl, isoxadifen-ethyl, fenchlorazole-ethyl, MG 191 or combinations thereof.

Herbicides of the subject invention include, but are not limited to, herbicides that inhibit pigment biosynthesis and/or plant light processes, including but not limited to, herbicides that inhibit protoporphoryinogen IX oxidase (Protox), herbicides that inhibit carotenoid biosynthesis (e.g. inhibitors of p-hydroxyphenylpyruvate dioxygenase, phytoene desaturase, others), herbicides that inhibit Photosystem I in photosynthesis (including bypyridilium herbicides that target ferredoxin-mediated metabolic reactions), and herbicides that inhibit Photosystem II in photosynthesis (e.g. herbicides that target multiple bindings of the Qb binding niche of the D1 protein); herbicides that impact plant growth and development, including but not limited to, herbicides that inhibit amino acid formation and conversion (e.g. inhibitors of acetolactate synthase, 5-enolpyruvylshikimate-3-phosphate synthase, or giutamine synthetase), herbicides that inhibit microtubule organization or assembly in mitosis, herbicides that inhibit the formation of very long chain fatty acids, herbicides that inhibit cellulose biosynthesis, and herbicides that inhibit auxin transport, and through mimicry of internal auxins.

Another aspect of the invention involves applications of herbicide inhibitors of photosynthesis alone and in mixtures with one or more herbicides with or without diluents, adjuvants, fertilizers, nutrient based solutions, other agrochemicals, crop safeners and/or other additives in order to increase the levels or to change the ratio of one or more phytonutrients in plants. Inhibitors of photosynthesis include those which target the following sites on the Qb binding niche of the D1 protein in photosystem II of photosynthesis such as 1) site A group 5(Cl) (examples include, but are not limited to ametryn, bromacil, desmedipham, phenmedipham, hexazinone, atrazine, pyrazon, simazine, metribuzin, prometryn, prometon, amicarbazone and terbacil); 2) site B group 6(C3) (examples include, but are not limited to bentazon, bromoxynil, ioxynil and pyridate; 3) site A 7(C2) [which bind to this site in a different fashion as compared to group 5(C1)] (examples include, but are not limited to propanil, diuron, linuron, siduron, fluometuron and tebuthiuron). Also, included are inhibitors of Photosystem I in photosynthesis (targeting ferrodoxin-mediated metabolic reactions) which are the bipyridilium herbicides paraquat and diquat. In addition, natural products which inhibit photosynthesis, such as sorgoleone, fischerellin A, juglone, anthroquinone and capsaicin can be applied to plants to enhance phytonutrient production or genetic modifications can be made to plant pathways altering levels of these materials produced in plants in order to increase or to change the ratio of phytonutrients in plants.

Another aspect of this invention involves applications of herbicides that are responsible for the conversion or inhibition of amino acid synthesis and/or conversion in plants. These inhibitors include herbicides which target either acetolactate synthase (ALS), 5-enolpyruvylshikimate-3-phosphate (EPSP) synthase, or glutamine synthetase (GS) alone and in mixtures with one or more herbicides with or without diluents, adjuvants, fertilizers, nutrient based solutions, other agrochemicals, crop safeners and/or other additives in order to increase the levels or to change the ratio of one or more phytonutrients in plants. Inhibitors of ALS include multiple chemical classes such as: 1) imidazolinones (examples include, but are not limited to imazethapyr, imazapyr, imazaquin, imazamox imazamethabenz, and imazapic); 2) pyrimidinylthio-benzoates (examples include, but are not limited to byspyribac and pyrithiobac); 3) sulfonylamino-carboynytriazolinones (examples include but are not limited to flucarbazone, thiencarbazone and propoxycarbazone); 4) sulfonylureas (examples include, but are not limited to amidosulfuron, azimsulfuron, bensulfuron, chlorimuron, chlorsulfuron, ethoxysulfuron, flazasulfuron, flupyrsulfuron, foramsulfuron, halosulfuron, imazosulfuron, iodosulfuron, mesosulfuron, nicosulfuron, primisulfuron, rimsulfuron, sulfosulfuron, sulfometuron, thifensulfuron, tribenuron, trifloxysulfuron and triflusulfuron); and/or 5) triazolopyrimidines (examples include, but are not limited to cloransulam, florasulam, flumetsuam, penoxsulam, pyroxsulam, and diclosulam). One commercially available inhibitor of EPSP synthase is glyphosate which is or has been sold in various formulations with different salts and esters (i.e., potassium, trimethysulfonium, isopropylamine, diammonium, or other salts and esters). In addition, natural products which inhibit ALS, GS, or EPSP synthase, such as gliotoxin or bialaphos, can be applied to plants to enhance phytonutrient production or genetic modifications can be made to plant pathways altering levels of these materials produced in plants in order to increase or to change the ratio of phytonutrients in plants.

Another aspect of this invention involves applications of herbicides that are responsible for inhibition of pigment biosynthesis in plants with one or more herbicides with or without diluents, adjuvants, fertilizers, nutrient based solutions, other agrochemicals, crop safeners and/or other additives in order to increase the levels or to change the ratio of phytonutrients in plants. Specific pigments include those herbicides that inhibit carotenoid biosynthesis and chlorophyll biosynthesis. Enzymes responsible for carotenoid biosynthesis include deoxy-D-xylulose-5-phosphate reductoisomerase (DOXP reductoisomerase), p-hydroxyphenylpyruvate dioxygenase (HPPD), zeta-carotene desaturase (ZDS), 1-deoxy-D-xylulose-5-phosphate synthatase (DOXP synthase), lycopene cyclase (LC), and phytoene desaturase (PDS), p-hydroxyphenylpyruvate dioxygenase, among others. Fosmidomycin, an antiobiotic drug, is an example of a herbicide inhibitor of DOXP reductoisomerase. HPPD inhibitor examples include, but are not limited to bicyclopyrone, mesotrione, sulcotrione, tembotrione, isoxaflutole, isoxachlortole, topramezone, benzofenap, pyrasulfatole, and pyrazoxyfen. ZDS inhibitors include, but are not limited to DFPC. DOXP synthase inhibitor examples include, but are not limited to clomazone. Herbicide inhibitor examples of PDS include but are not limited to norflurazon, flurochloridone, diflufenican, fluridone, picolinafen, flurtamone, and beflubutamid. Inhibitors of LC include but are not limited to amitrole and CPTA. An enzyme responsible for chlorophyll biosynthesis is protoporphyrinogen IX oxidase (PPO or Protox) which is inhibited by herbicides included in but not limited to the following chemical families: 1) diphenylethers (examples include, but are not limited to acifluorfen, lactofen, oxyfluorfen, and fomesafen); 2) triazolinones (examples include, but are not limited to carfentrazone, sulfentrazone, and azafenidin; 3) oxadiazoles (examples include, but are not limited to oxadiazon and oxadiargyl); 4) N-phenyl-phthalimides (examples include, but are not limited to cinidon, flumioxazin, and flumiclorac; 5) phenylpyrazoles (examples include, but are not limited to pyraflufen and fluazolate); 6) pyrimidinediones (examples include, but are not limited to saflufenacil, butafenacil, other); 7) thiadiazoles (examples include, but are not limited to fluthiacet). In addition, natural products which inhibit pigment synthesis, such as leptospermone can be applied to plants to enhance phytonutrient production or genetic modifications can be made to plant pathways altering levels of these materials produced in plants in order to increase or to change the ratio of phytonutrients in plants.

An additional aspect of this invention relates to herbicide inhibitors that interfere with plant cellular growth through: 1) inhibition of microtubule organization or assembly in mitosis; 2) inhibition of lipid synthesis; 3) inhibition of very long chain fatty acids; 4) inhibition of cellulose biosynthesis; 5) inhibition of auxin transport; and 7) through mimicry of internal auxins. Inhibitors of microtubule assembly by impacting tubulin formation include: 1) dinitroanilines (examples include, but are but not limited to pendimethalin, trifluralin, oryzalin, prodiamine, benefin, ethalfluralin, other); 2) the benzoic acid herbicide DCPA; 3) phosphoroamidates (examples include, but are not limited to butamiphos and amiprophos); 4) benzamides (examples include, but are not limited to pronamide and tebutam). Inhibitors of microtubule organization include, but are not limited to the carbamates herbicides carbetamide, propham, and chlorpropham. Inhibitors of the lipid synthesis that target acetyl CoA carboxylase (ACCase) include: 1) the cyclohexanedione (Dims) herbicides (examples include, but are not limited to clethodim, sethoxydim, tralkoxydim, other); 2) the aryloxyphenoxy propionate herbicides (examples include, but are not limited to fluazifop, fenoxaprop, quizalofop, diclofop, haloxyfop, other); 3) phenylpyrazoles (examples include, but are to pinoxaden). Inhibitors of lipid synthesis that do not target ACCase include: 1) thiocarbamates (examples include, but are not limited to EPTC, pebulate, vernolate, molinate, triallate, butylate, other); 2) the phosphorodithioate herbicide bensulide; 3) the benzofurane herbicides ethofumesate and benfuresate; 4) the chloro carbonic acid herbicides TCA, dalapon, and flupropanate. Inhibitors of very long chain fatty acid biosynthesis include: 1) chloracetamides (examples include, but are not limited to metazachlor, metolachior, acetochlor, dimethenamid, pretilachlor, propachlor, alachlor, other); 2) acetamides (examples include, but are not limited to diphenamid, napropamide, and naproanilide); 3) oxyacetamides (examples include, but are not limited to flufenacet and mefenacet); 4) the tetrazolinone herbicide fentrazamide; 4) other herbicides that inhibit very long chain fatty acid biosynthesis include, but are not limited to anilofos, piperophos, cafenstrole, indanofan, fenoxasulfon, and pyroxasulfone. Inhibitors of celloluse biosynthesis include but are not limited to: 1) the nitriles dichlobenil and chlorthiamid; 2) the bezamide herbicide isoxaben; 3) the triazolocarboxamide herbicide flupoxam; 4) the fluoroalkytriazine (examples include, but are not limited to indaziflam). Inhibitors of auxin transport include diflufenzopyr and naptalam. Auxin mimic or synthetic auxin herbicides include: 1) pyridine carboxylic acids (examples include, but are not limited to picloram, fluroxypyr, clopyralid, aminopyralid, triclopyr, other; 2) pyrimidine carboxylic acid herbicides (examples include, but are not limited to aminocyclopyrachlor); 3) phenoxy carboxylic acids (examples include but are not limited to 2,4-D, 2,4-DB, 2,4,5-T, dichlorprop, mecoprop, MCPA, MCPB, MCPP, other); 4) the benzoic acid herbicides (examples include, but not limited to dicamba, tricamba, and chloramben); 5) the quinoline carboxylic acids (examples include, but are not limited to quinclorac and quinmerac). In addition, natural products which inhibit plant cellular growth, such as ailanthone and catechin, can be applied to plants to enhance phytonutrient production or genetic modifications can be made to plant pathways altering levels of these materials produced in plants in order to increase or to change the ratio of phytonutrients in plants.

Defoliants/desiccants of the subject invention include, but are not limited to dimethipin, endothall, pelargonic acid, sodium chlorate, thiadiazuron, and tribufos. In addition, the previously mentioned herbicides glufosinate, paraquat, diquat, pyraflufen, and carfentrazone are often classified as defoliants/desiccants when used as such. Defoliants/desiccants can be applied to plants in order to increase or the change the ratio of phytonutrients in plants.

Fungicides and/or antibiotics of the subject invention include, but are not limited to, those that inhibit nucleic acid synthesis; impact mitosis and/or cellular division; inhibit cellular respiration; inhibit amino acid and/or protein synthesis; inhibit lipids and/or membrane synthesis; impact cellular signal transduction; impact sterol biosynthesis in membranes; affect glucan synthesis; impact host-pathogen interactions, including but not limited to those chemicals that impact host plant defense responses; and any related anti-fungal, anti-bacterial, or anti-viral chemical currently with activity of unknown or uncertain modes of actions and/or multi-site contact activity.

Another aspect of this invention involves applications of fungicides and/or antibiotics that are responsible for inhibition of nucleic acid synthesis with one or more fungicides and/or antibiotics with or without diluents, adjuvants, fertilizers, nutrient based solutions, other agrochemicals, crop safeners and/or other additives. Examples of fungicides and/or antibiotics that impact nucleic acid synthesis are phenylamides (metalaxyl, mefenoxam, among others) and heteroaromatics (octhilinone, among others). The application of fungicides and/or antibiotics that impact mitosis and/or cellular divisions include, but are not limited to, methyl-benzimidazole-carbamate fungicides (carbendazim, thisbendazole, among others) and benzamides (zoxamide, among others). Applications of fungicides and/or antibiotics that impact respiration such as carboxamides (flutolanil, carboxin, oxycarboxin, boscalid, penthiopyrad, pen flufen, among others), quinone outside inhibitors (azoxystrobin, pyraclostrobin, kresoxim-methyl, trifloxystrobin, famoxadone, fluoxastrobin, fenamidone, picoxystrobin, pyraoxystrobin, pyrametostrobin, among others), uncouplers of oxidative phosphorylation (fluazinam, among others), organo tin compounds (fentin hydroxide, among others). The application of fungicides and/or antibiotics that impact amino acid and/or protein synthesis include, but are not limited to, anilino-pyrimidines (cyprodinil, pyrimethanil, among others), glucopyranosyl antibiotics (streptomycin, among others), and tetracycline antibiotics (oxytetracycline, among others). The application of fungicides and/or antibiotics that impact signal transduction events include, but are not limited to, quinolines (quinoxyfen, among others), phenylpyrroles (fludiosonil, among others), and dicarboximides (iprodione, vinclozolin, among others). The application of fungicides and/or antibiotics that impact lipids and/or membrane synthesis include, but are not limited to, aromatic hydrocarbons (chloroneb, dicloran, quintozene, among others), heteroaromatics (etridiazole among others), carbamates (propamocarb, among others), and carboxylic acid amides (dimethomorph, mandipropamid, among others). The application of fungicides and/or antibiotics that impact sterol biosynthesis in membranes include, but are not limited to, demethylation inhibitors (triforine, fenarimol, imazalil, triflumizole, difenoconazole, fenbuconazole, ipconazole, metconazole, myclobutanil, propiconazole, prothioconazole, tebuconazole, tetraconazole, triadimefon, tridimenol, among others), amides (piperalin, among others), and hydroxyanilides (fenhexamid, among others). The application of fungicides and/or antibiotics that impact glucan synthesis include, but are not limited to, polyoxins (polyoxin, among others). The application of fungicides and/or antibiotics that impact host plant defense induction include, but are not limited to, benzo-thiodiazole (acibenzolar-s-methyl, among others). The application of fungicides and/or antibiotics currently with unknown modes of action include, but are not limited to, cyanoacetamide-oxime (cymoxanil, among others), phosphonates (fosetyl-Al, phosphorous acid and salts, among others), mineral oils, organic oils, potassium compounds, bicarbonates, and materials of biological origins. The application of fungicides and/or antibiotics with multi-site contact activity include, but are not limited to, inorganic salts (copper, copper salts, sulfur, among others), dithiocarbamates and related compounds (ferbam, mancozeb, maneb, metiram, thiram, ziram, among others), phthalimides (captan, folpet, among others), chloronitriles (chlrothalonil, among others), and guanidines (dodine, among others). The fungicides and/or antibiotics mentioned above, in addition to other related natural products can be applied to enhance phytonutrient production or to change the ratio of phytonutrients in plants.

Insecticides, nematicides, and/or miticides of the subject invention include, but are not limited to, those that are acetylcholinesterase inhibitors; act as GABA-gated chloride channel antagonists; are sodium channel modulators; act as nicotinic acetylcholine receptor agonists; are nicotinic acetylcholine receptor allosteric activators; are chloride channel activators; mimic juvenile hormones; act as miscellaneous non-specific, multi-site inhibitors; act as selective Homopteran feeding blockers; are mite growth inhibitors; insecticides that disrupt microbial activities of insect midgut membranes; act as inhibitors of mitochondrial ATP synthase; act as uncouplers of oxidative phosphorylation via disruption of the proton gradient; are nicotinic acetylcholine receptor channel blockers; are inhibitors of chitin biosynthesis (type 1); are molting disruptors (Dipteran); act as ecdysone receptor agonists; are octopamine receptor agonists; are inhibitors of mitochondrial complex III electron transport and mitochondrial complex I electron transport; block voltage-dependent sodium channels; are inhibitors of acetyl CoA carboxylase; are inhibitors of mitochondrial complex IV electron transport and mitochondrial complex II electron transport; modulate ryanodine receptors; and any related insecticidal, nematicidal, or miticidal chemical currently with activity of unknown or uncertain modes of actions and/or multi-site contact activity.

Another aspect of this invention involves applications of insecticides that are responsible for inhibition of acetylcholinesterase with or without diluents, adjuvants, fertilizers, nutrient based solutions, other agrochemicals, crop safeners and/or other additives. Examples of insecticides, miticides, and/or nematicides that inhibition of acetylcholinesterase are carbamates (alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC, xylylcarb, among others) and organophophates (acephate, azamethiphos, azinphos-ethyl, azinphos-methyl, cadusafos, chlorethoxyfos, chlorfenvinphos, chlormephos, chlorpyrifos, chlorpyrifos-methyl, coumaphos, cyanophos, demeton-s-methyl, diazinon, dichlorvos/ DDVP, dicrotophos, dirnethoate, dimethylvinphos, disulfoton, EPN, ethion, ethoprophos, famphur, fenamiphos, fenitrothion, fenthion, fosthiazate, heptenophos, imicyfos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos- methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion, among others). Examples of insecticides miticides, and/or nematicides that act as antagonists of GABA-gated chloride channels include, but are not limited to, cyclodiene organochlorine (chlordane, endosulfan, among others) and phenylpyrazoles (ethiprole, fipronil, among others). Applications of insecticides miticides, and/or nematicides that modulate sodium channels include, but or not limited to, pyrethroids and/or pyrethrins (acrinathrin, allethrin, d-cis-trans allethrin, d-trans allethrin, bifenthrin, bioallethrin, bioallethrin s-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, permethrin, phenothrin, prallethrin, pyrethrin, resmethrin, RU 15525, silafluofen, tefluthrin, tetramethrin, tetramethrin, tralomethrin, transfluthrin, ZXI 8901, among others) and DDT, methoxychlor. Applications of insecticides, miticides, and/or nematicides that act as nicotinic acetylcholine receptor agonists include, but or not limited to, neonicotinoids (acetamiprid, clothianidin, dinotefuran, imidacloprid, nitenpyram, thiacloprid, thiamethoxam, among others) and nicotine. Applications of insecticides, miticides, and/or nematicides that act as nicotinic acetylcholine receptor allosteric activators include, but or not limited to, spinosyns (spinetoram, spinosad, among others). Applications of insecticides, miticides, and/or nematicides that act as chloride channel activators include, but or not limited to, avermectins and milbenycins (abamectin, emamectin benzoate, milbemectin, among others). Applications of insecticides, miticides, and/or nematicides that mimic juvenile hormones include, but or not limited to, juvenile hormone analogues (hydroprene, kinoprene, methoprene, among others), fenoxycarb, and pyriproxyfen. Applications of insecticides, miticides, and/or nematicides that inhibit miscellaneous non-specific sites and/or multi-sites include, but or not limited to, alkyl halides (methyl bromide, methyl iodide, and other alkyl halides), chloropicrin, sulfuryl fluoride, borax, and tartar emetic. Applications of insecticides, miticides, and/or nematicides that block selective Homopteran feeding include, but or not limited to, pymetrozine and flonicamind, among others. Applications of insecticides, miticides, and/or nematicides that act to inhibit mite growth include, but or not limited to, clofentexine, hexythiazox, and etoxazole. Applications of insecticides that act as microbial disruptors of insect midgut membranes include, but or not limited to, Bacillus thuringiensis and Bacillus sphaericus (Bacillus thuringiensis subsp. israelensis, Bacillus sphaericus, Bacillus thuringiensis subsp. aizawai, Bacillus thuringiensis subsp. kurstaki, Bacillus thuringiensis subsp. tenebrionis, among others) and Bt crop proteins (Cry1Ab, Cry1Ac, Cry1Fa, Cry2Ab, mCry3A, Cry3Ab, Cry3Bb, Cry34/35Ab1). Applications of insecticides miticides, and/or nematicides that act to inhibit mitochondrial STP synthase include, but or not limited to, diafenthiuron, organotin miticides (azocyclotin, cyhexatin, fenbutatin oxide, among others), propargite, and tetradifon. Applications of insecticides, miticides, and/or nematicides that act as uncouplers of oxidative phosphorilation via disruption of the proton gradient include, but or not limited to, chlorfenapyr and DNOC. Applications of insecticides, miticides, and/or nematicides that block nicotinic acetylcholine receptor channels include, but or not limited to, nereistoxin analogues (bensultap, cartap hydrochloride, thiocyclam, thiosultap-sodium, among others). Applications of insecticides, miticides, and/or nematicides that inhibit chitin biosynthesis (type 0) include, but or not limited to, benzoylureas (bistrifluron, chlorfluazuron, diflubenzuron, flucycloxuron, flufenoxuron, hexaflumuron, lufenuron, novaluron, noviflumuron, feflubenzuron, triflumuron, among others). Applications of insecticides, miticides, and/or nematicides that inhibit chitin biosynthesis (type 1) include, but or not limited to, buprofezin. Applications of insecticides, miticides, and/or nematicides that act as molting disruptors (Dipteran) include, but or not limited to, cyromazine. Applications of insecticides, miticides, and/or nematicides that act as ecdysone receptor agonists include, but or not limited to, diacylhydrazines (chromafenozide, halofenozide, methoxyfenozide, tebufenozide, among others). Applications of insecticides, miticides, and/or nematicides that act as octopamine receptor agonists include, but or not limited to, amitraz. Applications of insecticides, miticides, and/or nematicides that act as inhibitors of mitochondrial complex I electron transport include, but or not limited to, METI acaricides and insecticides (fenazaquin, fenpyroximate, pyrimidifen, pyridaben, tebufenpyrad, tolfenpyrad, among others) and rotenone. Applications of insecticides, miticides, and/or nematicides that act as inhibitors of mitochondrial complex II electron transport include, but or not limited to, cyenopyrafen. Applications of insecticides, miticides, and/or nematicides that act as inhibitors of mitochondrial complex III electron transport include, but or not limited to, hydramethylnon, acequinocyl, and fluacrypyrim. Applications of insecticides, miticides, and/or nematicides that act as inhibitors of mitochondrial complex IV electron transport include, but or not limited to, phosphine (aluminum phosphide, calcium phosphide, phosphine, zinc phosphide, among others) and cyanide. Applications of insecticides, miticides, and/or nematicides that block voltage-dependent sodium channels include, but or not limited to, indoxacarb and metaflumizone. Applications of insecticides, miticides, and/or nematicides that act as inhibitors of acetyl CoA carboxylase include, but or not limited to, tetronic and tetramic acid derivatives (spirodiclofen, spiromesifen, spirotetramat, among others). Applications of insecticides miticides, and/or nematicides that act as modulators of ryanodine receptors include, but or not limited to, diamides (chlorantraniliprole, cyantraniliprole, flubendiamide, among others). Applications of insecticidal, miticides, and/or nematicides compounds of unknown or uncertain modes of action include, but or not limited to, azadirachtin, benzoximate, bifenazate, chinomethionat, cryolite, dicothl, pyridalyl, and cyflumetafen. Insecticides, miticides, and/or nematicides mentioned above, in addition to other insecticidal, miticidal, and/or nematicidal natural products can be applied to enhance phytonutrient production or to change the ratio of phytonutrients in plants.

Plant Growth Regulators (PGRs) of the subject invention include, but are not limited to, PGRs that suppress levels of gibberellic acid and PGRs that increase ethylene concentrations in plants, PGRs that impact shoot elongation, PGRs that stimulate cell division and cell elongation, PGRs that affect ripening in fruits and vegetables, and PGRs which can slow and/or stop plant growth and development.

Another aspect of this invention involves applications of PGRs that impact plant growth and development with one or more PGRs with or without diluents, adjuvants, fertilizers, nutrient based solutions, other agrochemicals, crop safeners and/or other additives. Examples of PGRs that impact plant growth, development, and/or cellular activities are those with active ingredients such as ancymidol, butralin, alcohols, chlormequat chloride, cytokinins, daminozide, ethephon, ethylene, gibberellic acid and/or gibberellin mixtures, indole-3-butyric acid (IBA), maleic hydrazide and/or other potassium salts, mefluidide, mepiquat chloride and/or mepiquat pentaborate, naphthalene-acetic acid, 1-naphthaleneacetamide, n-decanol, paclobutrazol, prohexadione calcium, trinexapac-ethyl, and uniconazole. PGRs mentioned above, in addition to other natural products PGRs can be applied to enhance phytonutrient production or to change the ratio of phytonutrients in plants.

In some aspects of the invention, herbicides, fungicides, insecticides, nematicides, miticides, defoliants/desiccants, antibiotics, and/or plant growth regulators are applied at rates (in amounts) that cause visual injury of between about 1% to about 50%; about 5% to about 40%; or about 10% to about 30%. Visual injury is determined by estimating the average percentage of stunting, changes in tissue appearance (i.e., chlorosis, necrosis, bleaching, other), leaf drop, epinasty (twisting), lodging, root damage (i.e., pruned roots, fused roots, other), stem or petiole cracking, wilting, and/or other visible signs of plant stress that occurs to a treated plant as compared to untreated plants. Visual injury may be observed within 1 hour after treatment throughout the life of the plant, but is preferably observed within 3 hours to 1 year after application, more preferably observed between 12 hours to 6 months after application, and most preferably observed between 1 day to 3 months after application and this level of is injury is dependent on the application rates and formulations of the herbicides, fungicides, insecticides, nematicides, miticides, defoliants/desiccants, antibiotics, and/or plant growth regulators applied alone or in combinations together and what additives, adjuvants, fertilizers and other nutrient based solutions, safeners, and other agrochemicals might be applied in these types of mixtures. In addition, herbicides, fungicides, insecticides, nematicides, miticides, defoliants/desiccants, antibiotics, and/or plant growth regulators may not produce obvious visual injury to one not skilled in the art of this invention. Injury may also be measured with tools used to measure differences in plant height, plant weight (i.e., crop yield or biomass accumulation), root growth, chlorophyll fluorescence, photosynthesis, transpiration, electrical conductivity, pH (i.e., plant parts, sap, assimilates, etc.) or other quantitative measurements of plant health by comparing treated plants with untreated plants.

As would be apparent to one skilled in the art, the herbicides, fungicides, insecticides, nematicides, miticides, defoliants/desiccants, antibiotics, plant growth regulators, and other inhibitors discussed above could be simultaneously applied in a tank mixtures comprising two or more inhibitors or, alternatively, individual/sequential application of single inhibitor compositions can be applied to a plant. The individual/sequential application of these compositions can be separated by a period of time ranging from approximately 1 hour to approximately 1 year.

The application of a single safener, herbicide, fungicide, insecticide, nematicide, miticide, defoliant/desiccant, antibiotic, or plant growth regulator, or mixtures of multiple herbicides, fungicides, insecticides, nematicides, miticides, defoliants/desiccants, antibiotics, and/or plant growth regulators, or sequential applications of several safeners, herbicides, fungicides, insecticides, nematicides, miticides, defoliants/desiccants, antibiotics, and/or plant growth regulators may impact the quantity and quality of one or multiple phytonutrients, enhance the production of one or more anti-oxidant compounds, and/or improve plant stress tolerance, even those phytonutrients and/or anti-oxidant compounds created in, or related to, separate biosynthetic pathways. In addition, plants treated with a single safener, herbicide, fungicide, insecticide, nematicide, miticide, defoliant/desiccant, antibiotic, and/or plant growth regulator or a mixture of multiple safeners, herbicides, fungicides, insecticides, nematicides, miticides, defoliants/desiccants, antibiotics, and/or plant growth regulators may only improve the quantity or quality of a single phytonutrient or production of a single anti-oxidant compound. These improvements in phytonutrient quality and/or quantity, anti-oxidant compound production, and/or plant stress tolerance may or may not be related to the biosynthetic pathway associated with the production of the phytonutrient and/or anti-oxidant compounds, and/or the pathway inhibited, up-regulated, or indirectly impacted by the herbicides, fungicides, insecticides, nematicides, miticides, defoliants/desiccants, antibiotics, and/or plant growth regulators, or mixture thereof applied.

A further aspect of the invention involves a method for identifying a composition that improves the production of a phytonutrient of interest, comprising:

a) applying a candidate composition to a plant;

b) growing the plant that is treated with the candidate composition and harvesting at least part of the plant;

c) determining a level of a phytonutrient of interest in the harvested part of the treated plant;

d) comparing said level with that of a plant untreated with the candidate composition; and

e) identifying the candidate composition as a composition that improves the production of the phytonutrient of interest, if the level of the phytonutrient in the harvested part of the treated plant is higher than that of the plant untreated with the candidate composition.

The candidate composition of the subject invention encompasses an isolated chemical compound, a mixture of compounds, and a chemical formulation. In certain embodiments, the candidate composition includes a herbicide, an active ingredient of a herbicidal composition, and a mixture thereof, including for example, herbicides that inhibit photosynthesis, herbicides that inhibit protoporphoryinogen IX oxidase (Protox), herbicides that inhibit Photosystem I in photosynthesis, herbicides that inhibit Photosystem II in photosynthesis, herbicides that impact plant growth and development, herbicides that inhibit amino acid formation and conversion, herbicides that inhibit microtubule organization or assembly in mitosis, herbicides that inhibit the formation of very long chain fatty acids, herbicides that inhibit cellulose biosynthesis, and herbicides that inhibit auxin transport. In additional embodiments, the candidate composition includes a defoliant/desiccant, an active ingredient of a defoliating/desiccating composition, and a mixture thereof, including the active ingredients dimethipin, endothall, pelargonic acid, sodium chlorate, thiadiazuron, tribufos, glufosinate, paraquat, diquat, pyraflufen, and carfentrazone. In additional embodiments, the candidate composition includes a fungicide and/or antibiotic, an active ingredient of a fungicidal, bactericidal, and/or antimicrobial composition, and a mixture thereof, including but not limited to, those that inhibit nucleic acid synthesis; impact mitosis and/or cellular division; inhibit cellular respiration; inhibit amino acid and/or protein synthesis; inhibit lipids and/or membrane synthesis; impact cellular signal transduction; impact sterol biosynthesis in membranes; affect glucan synthesis; impact host-pathogen interactions, including but not limited to those chemicals that impact host plant defense responses; and any related anti-fungal, anti-bacterial, or anti-viral chemical currently with activity of unknown or uncertain modes of actions and/or multi-site contact activity. In additional embodiments, the candidate composition includes an insecticide, nematicide, and/or miticide, an active ingredient of insecticidal, nematicidal, miticidal, and/or acaricidal composition, and a mixture thereof, including but not limited to, those that are acetylcholinesterase inhibitors; act as GABA-gated chloride channel antagonists; are sodium channel modulators; act as nicotinic acetylcholine receptor agonists; are nicotinic acetylcholine receptor allosteric activators; are chloride channel activators; mimic juvenile hormones; act as miscellaneous non-specific, multi-site inhibitors; act as selective Homopteran feeding blockers; are mite growth inhibitors; insecticides that disrupt microbial activities of insect midgut membranes; act as inhibitors of mitochondrial ATP synthase; act as uncouplers of oxidative phosphorylation via disruption of the proton gradient; are nicotinic acetylcholine receptor channel blockers; are inhibitors of chitin biosynthesis (type 1); are molting disruptors (Dipteran); act as ecdysone receptor agonists; are octopamine receptor agonists; are inhibitors of mitochondrial complex III electron transport and mitochondrial complex I electron transport; block voltage-dependent sodium channels; are inhibitors of acetyl CoA carboxylase; are inhibitors of mitochondrial complex IV electron transport and mitochondrial complex II electron transport; modulate ryanodine receptors; and any related insecticidal, nematicidal, or miticidal chemical currently with activity of unknown or uncertain modes of actions and/or multi-site contact activity. In additional embodiments, the candidate composition includes a plant growth regulator, an active ingredient of a plant growth regulating composition, and a mixture thereof, including but not limited to, PGRs that suppress levels of gibberellic acid and PGRs that increase ethylene concentrations in plants, PGRs that impact shoot elongation, PGRs that stimulate cell division and cell elongation, PGRs that affect ripening in fruits and vegetables, and PGRs which can slow and/or stop plant growth and development.

In certain embodiments, the candidate composition further comprises diluents, adjuvants, fertilizers, nutrient based solutions, agrochemicals, crop safeners and/or other additives.

In certain specific embodiments, said one or more herbicides, defoliants/desiccants, insecticides, nematicides, miticides, fungicides, antibiotics, plant growth regulators have a mode of action selected from the following modes, sites or mechanisms of action and/or plant processes by which these agrochemicals or pesticides either inhibit, activate, modulate, uncouple, up-regulate, mimic, disrupt, or otherwise modify. These targets include protoporphoryinogen IX oxidase (Protox), carotenoid biosynthesis [e.g. inhibitors of phytoene desaturase, deoxy-D-xylulose-5-phosphate reductoisomerase (DOXP reductoisomerase), p-hydroxyphenylpyruvate dioxygenase (HPPD), zeta-carotene desaturase (ZDS), 1-deoxy-D-xylulose-5-phosphate synthatase (DOXP synthase), lycopene cyclase (LC), and phytoene desaturase (PDS), Photosystem I in photosynthesis (targeting ferredoxin-mediated metabolic reactions), Photosystem II in photosynthesis (multiple bindings of the Qb binding niche of the D1 protein), inhibitors of acetolactate synthase, 5-enolpyruvylshikimate-3-phosphate synthase, giutamine synthetase microtubule organization or assembly in mitosis, formation of very long chain fatty acids, cellulose biosynthesis, auxin transport, internal auxins, nucleic acid synthesis; mitosis and/or cellular division; cellular respiration; amino acid and/or protein synthesis; lipids and/or membrane synthesis, cellular signal transduction, sterol biosynthesis, glucan synthesis, host-pathogen interactions, acetylcholinesterase, GABA-gated chloride channel antagonists, sodium channel modulators, nicotinic acetylcholine receptor agonists, nicotinic acetylcholine receptor allosteric activators, chloride channel activators, mimic juvenile hormones, Homopteran feeding blockers, mite growth, activities of insect midgut membranes, mitochondrial ATP synthase, oxidative phosphorylation via proton gradient, nicotinic acetylcholine receptor channel blockers, chitin biosynthesis (type 1), molting disruptors (Dipteran), ecdysone receptor agonists, octopamine receptor agonists, mitochondrial complex III electron transport and mitochondrial complex I electron transport, voltage-dependent sodium channels, acetyl CoA carboxylase, mitochondrial complex IV electron transport and mitochondrial complex II electron transport, ryanodine receptors, gibberellic acid production, ethylene production, shoot elongation, cell division and cell elongation, ripening, overall plant growth and development. Other agrochemical or pesticide modes, sites, or mechanisms of action also apply to this embodiment and a complete list of them can be found on the websites of the “Herbicide Resistance Action Committee or HRAC”, the “Fungicide Resistance Action Committtee or FRAC”, and the “Insecticide Resistance Action Committee or IRAC”.

In certain specific embodiments, the candidate compositions may be herbicides, defoliants/desiccants, insecticides, nematicides, miticides, fungicides, antibiotics, plant growth regulators, is selected from a group consisting of ametryn, bromacil, desmedipham, phenmedipham, hexazinone, atrazine, pyrazon, simazine, metribuzin, prometryn, prometon, amicarbazone, terbacil, bentazon, bromoxynil, ioxynil, pyridate, propanil, diuron, linuron, siduron, fluometuron, tebuthiuron, paraquat, diquat, sorgoleone, fischerellin A, juglone, anthroquinone, capsaicin, imazethapyr, imazapyr, imazaquin, imazamox imazamethabenz, imazapic, byspyribac, pyrithiobac, flucarbazone, thiencarbazone, propoxycarbazone, amidosulfuron, azimsulfuron, bensulfuron, chlorimuron, chlorsulfuron, ethoxysulfuron, flazasulfuron, flupyrsulfuron, foramsulfuron, halosulfuron, imazosulfuron, iodosulfuron, mesosulfuron, nicosulfuron, primisulfuron, rimsulfuron, sulfosulfuron, sulfometuron. thifensulfuron, tribenuron, trifloxysulfuron, triflusulfuron, cloransulam, florasulam, flumetsuam, penoxsulam, pyroxsulam, diclosulam, glyphosate, gliotoxin, bialaphos, fosmidomycin, mesotrione, sulcotrione, tembotrione, isoxaflutole, bicyclopyrone, pyrasulfatole, isoxachlortole, topramezone, benzofenap, pyrazoxyfen, DFPC, clomazone, norflurazon, flurochloridone, diflufenican, fluridone, picolinafen, flurtamone, beflubutamid, amitrole, CPTA acifluorfen, lactofen, oxyfluorfen, fomesafen, carfentrazone, sulfentrazone, azafenidin oxadiazon, oxadiargyl, cinidon, flumioxazin, flumiclorac, pyraflufen, fluazolate, saflufenacil, butafenacil, fluthiacet, leptospermone, pendimethalin, trifluralin, oryzalin, prodiamine, benefin, ethalfluralin, DCPA, butamiphos, amiprophos, pronamide, tebutam, carbetamide, propham, chlorpropham, clethodim, sethoxydim, tralkoxydim, fluazifop, fenoxaprop, quizalofop, diclofop, haloxyfop, pinoxaden, EPTC, pebulate, vernolate, molinate, triallate, butylate, bensulide, ethofumesate, benfuresate, TCA, dalapon, flupropanate, metazachlor, metolachlor, acetochlor, dimethenamid, pretilachlor, propachlor, alachlor, diphenamid, napropamide, naproanilide, flufenacet, mefenacet, fentrazamide, anilofos, piperophos, cafenstrole, indanofan, pyroxasulfone, dichlobenil, chlorthiamid, isoxaben, flupoxam, indaziflam, diflufenzopyr, naptalam, picloram, fluroxypyr, clopyralid, aminopyralid, triclopyr, aminocyclopyrachlor, 2,4-D, 2,4-DB, 2,4,5-T, dichlorprop, mecoprop, MCPA, MCPB, MCPP, dicamba, tricamba, chloramben, quinclorac, quinmerac, ailanthone, catechin, dimethipin, endothall, pelargonic acid, sodium chlorate, thiadiazuron, tribufos metalaxyl, mefenoxam, octhilinone, carbendazim, thisbendazole, zoxamide, flutolanil, carboxin, oxycarboxin, boscalid, azoxystrobin, pyraclostrobin, kresoxim-methyl, trifloxystrobin, famoxadone, fluoxastrobin, fenamidone, fluazinam, fentin hydroxide, cyprodinil, pyrimethanil, streptomycin, tetracycline, oxytetracycline, quinoxyfen, fludiosonil, iprodione, vinclozolin, chloroneb, dicloran, quintozene, etridiazole, propamocarb, dimethomorph, mandipropamid, triforine, fenarimol, imazalil, triflumizole, difenoconazole, fenbuconazole, ipconazole, metconazole, myclobutanil, propiconazole, prothioconazole, tebuconazole, tetraconazole, triadimefon, tridimenol, piperalin, fenhexamid, polyoxin, acibenzolar-s-methyl, cymoxanil, fosetyl-Al, phosphorous acid and salts, mineral oils, organic oils, potassium compounds, bicarbonates, copper, copper salts, sulfur, ferbam, mancozeb, maneb, metiram, thiram, ziram, captan, folpet, chlrothalonil, dodine, alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC, xylylcarb, acephate, azamethiphos, azinphos-ethyl, azinphos-methyl, cadusafos, chlorethoxyfos, ehlorfenvinphos, chlormephos, chlorpyrifos, chlorpyrifos-methyl, coumaphos, cyanophos, demeton-s-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyfos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos- methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion, chlordane, endosulfan, ethiprole, fipronil, acrinathrin, allethrin, d-cis-trans allethrin, d-trans allethrin, bifenthrin, bioallethrin, bioallethrin s-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, permethrin, phenothrin, prallethrin, pyrethrin, resmethrin, RU 15525, silafluofen, tefluthrin, tetramethrin, tetramethrin, tralomethrin, transfluthrin, ZXI 8901, DDT, methoxychlor, acetamiprid, clothianidin, dinotefuran, imidacloprid, nitenpyram, thiacloprid, thiamethoxam, nicotine, spinetoram, spinosad, abamectin, emamectin benzoate, milbemeetin, hydroprene, kinoprene, methoprene, fenoxycarb, pyriproxyfen, methyl bromide, methyl iodide, chloropicrin, sulfuryl fluoride, borax, and tartar emetic, pymetrozine, flonicamind, clofentexine, hexythiazox, etoxazole, Bacillus thuringiensis, Bacillus sphaericus, Bacillus thuringiensis subsp. israelensis, Bacillus sphaericus, Bacillus thuringiensis subsp. aizawai, Bacillus thuringiensis subsp. kurstaki, Bacillus thuringiensis subsp. tenebrionis, Cry1Ab, Cry1Ac, Cry1Fa, Cry2Ab, mCry3A, Cry3Ab, Cry3Bb, Cry34/35Ab1, diafenthiuron, azocyclotin, cyhexatin, fenbutatin oxide, propargite, tetradifon, chlorfenapyr, DNOC, bensultap, cartap hydrochloride, thiocyclam, thiosultap-sodium, bistrifluron, chlorfluazuron, diflubenzuron, flucycloxuron, flufenoxuron, hexaflumuron, lufenuron, novaluron, noviflumuron, feflubenzuron, triflumuron, buprofezin, cyromazine, chromafenozide, halofenozide, methoxyfenozide, tebufenozide, amitraz, fenazaquin, fenpyroximate, pyrimidifen, pyridaben, tebufenpyrad, tolfenpyrad, rotenone, cyenopyrafen, hydramethylnon, acequinocyl, fluacrypyrim, phosphine, aluminum phosphide, calcium phosphide, phosphine, zinc phosphide, cyanide, indoxacarb, metaflumizone, spirodiclofen, spiromesi fen, spirotetramat, chlorantraniliprole, cyantraniliprole, flubendiamide, azadirachtin, benzoximate, bifenazate, chinomethionat, cryolite, dicofol, pyridalyl, cyflumetafen, ancymidol, butralin, alcohols, chlormequat chloride, cytokinins, daminozide, ethephon, ethylene, gibberellic acid and/or gibberellin mixtures, indole-3-butyric acid (IBA), maleic hydrazide and/or other potassium salts, mefluidide, mepiquat chloride and/or mepiquat pentaborate, naphthalene-acetic acid, 1-naphthaleneacetamide, n-decanol, paclobutrazol, prohexadione calcium, trinexapac-ethyl, uniconazole, fenoxasulfone, penthiopyrad, picoxystrobin, pyraoxystrobin, pyrametostrobin, penflufen and any combination thereof Other pesticides also apply to this embodiment and a complete list of pesticides that apply to this embodiment can be found at the “Compendium of Pesticide Common Names” which is hereby incorporated by reference in its entirety and can be located on the internet at World Wide Website: alanwood.net/pesticides/.

The phytonutrient of interest includes, but are not limited to, lipids (i.e., oils, fatty acids, saturated fatty acids, non-saturated fatty acids, steroids, other), vitamins [Vitamin A (retinol), Vitamin B₁ (thiamine), Vitamin B₂ (riboflavin), Vitamin B₃ (niacin), Vitamin B₁₂, Folic acid (folacin), Vitamin C (Ascorbic acid), Vitamin D, Vitamin E (tocopherols), Vitamin K, other], minerals (i.e. calcium, iron, iodide, fluoride, zinc, etc.), proteins, amino acids (i.e., histidine, valine, leucine, isoleucine, lysine, methionine, cysteine, phenylalanine, tyrosine, threonine, tryptophan, etc.), carbohydrates [including but not limited to starch, fiber, cellulose, and sugars (i.e., sucrose, fructose, glucose, maltose, raffanose, etc.)] carotenoid/xanthophyll antioxidants (i.e., beta-carotene, lycopene, lutein, zeaxanthin, antheraxanthin, etc.), glucosinolates (i.e., glucobrassicin, sinalbin, etc.) and phenolic compounds (i.e. capsaicin, eugenol, polyphenols, salicylic acid, anthocyanins, tannins, resveratrol, etc.), antioxidant compounds (e.g. terpenes (carotenoids and essential oils), phenolics (flavonoids and anthocyanins), nitrogen-containing compounds (alkaloids and glucosinolates)), and minerals (i.e. N, P, K, Ca, Mg, S, Cl, Co, Cu, Fe, Mn, Mo, Na, Ni, Zn, etc.).

Any part of the plant may be harvested for determining the level of phytonutrient production, including but not limited to, roots, flowers, barks, leaves, seeds, stems, petioles or fruits. For example, alfalfa, barley, corn (maize), popcorn, sweet corn, sorghum, cotton, soybean, sugarbeets, sunflower, sugarcane, rape, canola, peanuts, rice, oats, triticale, rye, agave, wheat, potato, tomato, fruits (i.e., apple, apricot, avocado, breadfruit, banana, blackberry, blackcurrant, blueberry, cherimoya, cherry, clementine, coconut, cranberry, durian, fig, grapefruit, grape, guava, jackfruit, kiwi, lemon, lime, loganberry, mandarin, mango, mangosteen, melon, nectarine, orange, papaya, peach, pear, persimmon, pineapple, plum, pomegranate, quince, satsuma, strawberry, tamarillo, ugli fruit, watermelon etc.), nuts (i.e., almond, beech, butternut, brazil nut, candlenut, cashew, chestnuts, colocynth, filbert. hickory, pecan, shagbark hickory, kola nut, macadamia, mamoncillo, maya nut, oak acorns, ogbono nut, paradise nut, pili nut, pistachio, walnut, etc.), vegetables (i.e., asparagus, artichoke, leafy greens, melons, snapbean, lima bean, cabbage, pea, spinach, pumpkin, onions, garlic, squash, eggplant, carrots, broccoli, sweet potato, zucchini, etc.), turfgrass (i.e., bahiagrass, bluegrass, buffalograss, fescue, bentgrass, bermudagrass, ryegrass, St. Augustinegrass, zoysiagrass, etc.), forage grasses, switchgrass, forage legumes (i.e., clover, lesepedeza, etc.), ornamental plants, forest plants (i.e., hardwood trees, pines, shrubs, vines, or wild flowers), herbs or other flavor enhancing crops (i.e., peppermint, spearmint, thyme, basil, coriander, dill, rosemary, Irish moss, arrowroot, sesame, etc.), and plantation crops (i.e., oil palm, cocoa, coffee, hops, pineapple, eucalyptus, etc.) can be analyzed for altered levels of phytonutirents. Additionally, the candidate composition of the present invention may be applied preplant, preemergence, postemergence, postemergence directed or as a layby application either continuously or intermittently. Crops that may be treated as disclosed herein may also be genetically modified crops, a cultivar produced through common methods of plant breeding (i.e., asexual propagation, chemical/radioactive induced mutations, pedigree breeding, backcrossing, etc.), or crops that have not been genetically modified or produced through common plant breeding techniques.

The candidate composition may be applied at a rate of 0.006 g ai/ha to approximately 6,000 g ai/ha, more preferred at a rate of 0.03 g ai/ha to 3,000 g ai/ha, and most preferred at a rate of 0.5 to 1,500 g ai/ha in a single or multiple applications. In alternative embodiments, the candidate composition may be applied at a rate of about 50 g ai/ha to 3000 g ai/ha, 100 g ai/ha to 2500 g ai/ha, 150 g ai/ha to 2250 g ai/ha, 200 g ai/ha to 2000 g ai/ha, 250 g ai/ha to 1750 g ai/ha, 300 g ai/ha to 1500 g ai/ha, 350 g ai/ha to 1250 g ai/ha, 400 g ai/ha to 1000 g ai/ha, 450 g ai/ha to 900 g ai/ha, 500 g ai/ha to 800 g ai/ha, or 600 g ai/ha to 700 g ai/ha, as would be apparent to one skilled in the art. Alternatively or additionally, the candidate composition may be applied at a rate to cause visual injury of between about 5% to about 45% about 15% to about 35%, about 20% to about 30% to the plant.

An additional embodiment of the invention provides a method for identifying a composition that improves plant stress tolerance to an abiotic stress condition of interest, comprising:

a) applying a candidate composition to a plant at a registered, elevated, or reduced application rates;

b) growing the plant that is treated with the candidate composition under an abiotic stress condition of interest;

c) determining a level of growth, development, weight, height, shoot generation, leaf area, root induction, bud formation, forage yield, flowering, and/or fruit generation of the plant treated with the candidate composition;

d) comparing said level with that of a plant untreated with the candidate composition growing under the abiotic stress condition of interest; and

e) identifying the candidate composition as a composition that improves the stress tolerance of the plant to the abiotic condition of interest, if the level of growth, development, weight, height, shoot generation, leaf area, root induction, bud formation, forage yield, flowering, and/or fruit generation of the plant treated with the candidate composition is higher than that of the plant untreated with the candidate composition.

The abiotic stress conditions of interest include, but are not limited to, drought, elevated temperatures, nutrient imbalances, applications of agrochemicals such as herbicides, fungicides, insecticides, nematicides, miticides, defoliants/desiccants, antibiotics, and/or plant growth regulators, and any combination thereof Unless specifically indicated or implied, the terms “a”, “an”, and “the” signify “at least one” as used herein. Additionally, the terms “comprising”, “consisting essentially of”, and “consisting of can be used interchangeably throughout the subject specification.

All patents, patent applications, provisional applications, and publications referred to or cited herein are incorporated by reference in their entirety to the extent they are not inconsistent with the explicit teachings of this specification. Following are examples which illustrate procedures for practicing the invention. These examples should not be construed as limiting.

### EXAMPLE 1

Studies were conducted to ascertain the tolerance and nutritional quality differences following postemergence applications of the ALS-inhibitor nicosulfuron and the HPPD-inhibitors mesotrione, tembotrione, and topramenzone alone and in mixtures together and with and without the crop safener isoxadifen-ethyl on the sweet corn (Zea mays var. rugosa) cultivar ‘Incredible’(yellow-kernel moderately sensitive genotype).

Herbicide treatments were applied in a water carrier at 23 gallons of solution per acre on a field site near Knoxville, Tenn. and Painter, Va. Treatments include the following: 1) nicosulfuron at 35 g ai/ha (Accent); 2) mesotrione at 105 g ai/ha (Callisto); 3) nicosulfuron at 35 g ai/ha+mesotrione at 105 g ai/ha (Callisto+Accent); 4) tembotrione at 92 g ai/ha+isoxadifen-ethyl at 46 g ai/ha (Laudis); 5) topramezone at 15 g ai/ha (Impact); 6) nicosulfuron at 35 g ai/ha+isoxadifen-ethyl at 9 g ai/ha (Accent Q); 7) nicosulfuron at 35 g ai/ha+isoxadifen-ethyl at 9 g ai/ha+topramezone at 15 g ai/ha (Accent Q+Impact); 8) nicosulfuron 35 g ai/ha+isoxadifen-ethyl at 9 g ai/ha+mesotrione at 105 g ai/ha (Accent Q+Callisto); 9) treated check (atrazine or Aatrex). All treatments included a PRE application of atrazine plus metolachlor in order to maintain weed free conditions. In addition, all POST treatments (including the treated check) included atrazine at 560 g ai/ha and crop oil concentrate at 1% v/v. Visual injury was rated on a percent scale from 0 to 100 where 0 equals no visual response and 100 equals complete plant death. Sweet corn was harvested from the center two rows of each four row plot and weighed in the husk. Eight ears of corn were harvested from each plot and were pooled over replicates for content analysis of crude protein, crude amino acid content, acid detergent fiber, neutral detergent fiber, total fatty acids, and key mineral content (including Na, Ca, P, Mg, K, Fe, Mn, Zn, and Cu). In addition, the amounts of key individual amino acids (aspartic acid, threonine, glutamic acid, proline, glycine, cysteine, valine, leucine, isoleucine, methionine, and lysine) and composite classifications of fatty acid (saturated fatty acids, monounsaturated fatty acids, polyunsaturated fatty acids) were also analyzed from these sub-samples. Phytonutrient amounts were converted to a percentage of the treated check to highlight the differences observed due to each herbicide(s) with or without safener application.

In general, injury was between 2 and 18% with all treatments by 7 days after treatment (data not presented). All treatments increased crude protein by 4 to 12% (Table 1). All treatments except for tembotrione plus isoxadifen-ethyl increased acid and neutral detergent fiber by 1 to 30%. Topramezone, mesotrione, nicosulfuron +isoxadifen-ethyl, and tembotrione plus isoxadifen-ethyl increased all amino acids by 2 to 28% (Table 2). These treatments were also especially valuable in increasing lysine (a deficient amino acid in corn) by 10 to 28%. With one exception, all treatments increased the mineral elements P, Mg, K, Na, Fe, Mn, and Zn by 1 to 75% (Table 3). In addition, the combination of nicosulfuron plus mesotrione plus isoxadifen-ethyl increased calcium by 100%. All measures of fatty acid content were improved by all treatments by 1 to 44% (Table 4). All treatments increased fructose and glucose 18 to 68% in comparison to the check. All treatments with the exception of nicosulfuron +isoxadifen-ethyl increased maltose by 4 to 23%. In addition, all treatments except mesotrione alone increased total sugars production by 9 to 16% in comparison to the check (Table 15). Finally, all treatment increased levels of the xanthophyll antioxidant by 14 to 49% over the check.” Regardless of the phytonutrient class evaluated, there were many obvious improvement in phytonutrient levels when isoxadifen-ethyl was added to nicosulfuron (Tables 1-4).

TABLE 1

| | | | | | | | Crop Name | | | |
|---|---|---|---|---|---|---|---|---|---|---|
| | | | | | | | Sweet corn | Sweet corn | Sweet corn | Sweet corn |
| | | | | | | | Crop Variety | | | |
| | | | | | | | Incredible | Incredible | Incredible | Incredible |
| | | | | | | | Rating Data Type | | | |
| | | | | | | | Protein | Amino Acid | Acid DF | Neutral DF |
| Trt | Treatment | Form | | Rate | Other | Other | Rating Unit | | | |
| No. | Name | Conc | Rate | Unit | Rate | Rate Unit | % of check | % of check | % of check | % of check |
| 1 | Nicosulfuron*ACCENT | 75 | 0.66 | oz/a | 0.031 | lb ai/a | 7 | −1 | 9 | 4 |
| | Atrazine*AATREX | 4 | 16 | oz/a | 1 | pt/a | | | | |
| 2 | Mesotrione*CALLISTO | 4 | 3 | oz/a | 0.094 | lb ai/a | 7 | 6 | 10 | 13 |
| | Atrazine*AATREX | 4 | 16 | oz/a | 1 | pt/a | | | | |
| 3 | Nicosulfuron*ACCENT | 75 | 0.66 | oz/a | 0.031 | lb ai/a | 8 | 7 | 5 | 1 |
| | Mesotrione*CALLISTO | 4 | 3 | oz/a | 0.094 | lb ai/a | | | | |
| | Atrazine*AATREX | 4 | 16 | oz/a | 1 | pt/a | | | | |
| 4 | Tembotrione*LAUDIS | 3.5 | 3 | oz/a | 0.082 | lb ai/a | 8 | 7 | 0 | 0 |
| | Atrazine*AATREX | 4 | 16 | az/a | 1 | pt/a | | | | |
| 5 | Topramezone*IMPACT | 2.8 | 0.75 | oz/a | 0.0164 | lb ai/a | 8 | 7 | 2 | 7 |
| | Atrazine*AATREX | 4 | 16 | oz/a | 1 | pt/a | | | | |
| 6 | Nicosulfuron*ACCENT Q | 54.5 | 0.9 | oz/a | 0.0307 | lb ai/a | 12 | 10 | 30 | 21 |
| | Atrazine*AATREX | 4 | 16 | oz/a | 1 | pt/a | | | | |
| 7 | Nicosulfuron*ACCENT Q | 54.5 | 0.9 | oz/a | 0.0307 | lb ai/a | 5 | 0 | 8 | 7 |
| | Topramezone*IMPACT | 2.8 | 0.75 | oz/a | 0.0164 | lb ai/a | | | | |
| | Atrazine*AATREX | 4 | 16 | oz/a | 1 | pt/a | | | | |
| 8 | Nicosulfuron*ACCENT Q | 54.5 | 0.9 | oz/a | 0.0307 | lb ai/a | 4 | 2 | 13 | 7 |
| | Mesotrione*CALLISTO | 4 | 3 | oz/a | 0.094 | lb ai/a | | | | |
| | Atrazine*AATREX | 4 | 16 | oz/a | 1 | pt/a | | | | |
| 9 | Atrazine*AATREX | 4 | 16 | oz/a | 1 | pt/a | 0 | 0 | 0 | 0 |

TABLE 2

| | | | | | | | Crop Name | | | | | |
|---|---|---|---|---|---|---|---|---|---|---|---|---|
| | | | | | | | Sweet | Sweet | Sweet | Sweet | Sweet | Sweet |
| | | | | | | | corn | corn | corn | corn | corn | corn |
| | | | | | | | Crop Variety | | | | | |
| | | | | | | | Incredible | Incredible | Incredible | Incredible | Incredible | Incredible |
| | | | | | | | Rating Data Type | | | | | |
| | | | | | | | Aspartic | | Glutamic | | | |
| | | | | | | | ac [symbol] | Threonine | ac [symbol] | Proline | Glycine | Cysteine |
| | | | | | | | Rating Unit | | | | | |
| Trt | Treatment | Form | | Rate | Other | Other | % | % | % | % | % | % |
| No. | Name | Conc | Rate | Unit | Rate | Rate Unit | of check | of check | of check | of check | of check | of check |
| 1 | Nicosulfuron*ACCENT | 75 | 0.66 | oz/a | 0.031 | lb ai/a | −4 | 2 | −2 | −7 | −4 | 0 |
| | Atrazine*AATREX | 4 | 16 | oz/a | 1 | pt/a | | | | | | |
| 2 | Mesotrione*CALLISTO | 4 | 3 | oz/a | 0.094 | lb ai/a | 7 | 16 | 6 | 9 | 4 | 6 |
| | Atrazine*AATREX | 4 | 16 | oz/a | 1 | pt/a | | | | | | |
| 3 | Nicosulfuron*ACCENT | 75 | 0.66 | oz/a | 0.031 | lb ai/a | 7 | 9 | 6 | 7 | −4 | 6 |
| | Mesotrione*CALLISTO | 4 | 3 | oz/a | 0.094 | lb ai/a | | | | | | |
| | Atrazine*AATREX | 4 | 16 | oz/a | 1 | pt/a | | | | | | |
| 4 | Tembotrione*LAUDIS | 3.5 | 3 | oz/a | 0.082 | lb ai/a | 5 | 11 | 8 | 7 | 6 | 9 |
| | Atrazine*AATREX | 4 | 16 | oz/a | 1 | pt/a | | | | | | |
| 5 | Topramezone*IMPACT | 2.8 | 0.75 | oz/a | 0.0164 | lb ai/a | 6 | 12 | 6 | 6 | 4 | 3 |
| | Atrazine*AATREX | 4 | 16 | oz/a | 1 | pt/a | | | | | | |
| 6 | Nicosulfuron*ACCENT Q | 54.5 | 0.9 | oz/a | 0.0307 | lb ai/a | 12 | 12 | 10 | 10 | 9 | 12 |
| | Atrazine*AATREX | 4 | 16 | oz/a | 1 | pt/a | | | | | | |
| 7 | Nicosulfuron*ACCENT Q | 54.5 | 0.9 | oz/a | 0.0307 | lb ai/a | 3 | 3 | 2 | 3 | 4 | −3 |
| | Topramezone*IMPACT | 2.8 | 0.75 | oz/a | 0.0164 | lb ai/a | | | | | | |
| | Atrazine*AATREX | 4 | 16 | oz/a | 1 | pt/a | | | | | | |
| 8 | Nicosulfuron*ACCENT Q | 54.5 | 0.9 | oz/a | 0.0307 | lb ai/a | −1 | 7 | 2 | 4 | 0 | 3 |
| | Mesotrione*CALLISTO | 4 | 3 | oz/a | 0.094 | lb ai/a | | | | | | |
| | Atrazine*AATREX | 4 | 16 | oz/a | 1 | pt/a | | | | | | |
| 9 | Atrazine*AATREX | 4 | 16 | oz/a | 1 | pt/a | 0 | 0 | 0 | 0 | 0 | 0 |
| | | | | | | | Crop Name | | | | | |
| | | | | | | | Sweet | Sweet | Sweet | Sweet | Sweet | Sweet |
| | | | | | | | corn | corn | corn | corn | corn | corn |
| | | | | | | | Crop Variety | | | | | |
| | | | | | | | Incredible | Incredible | Incredible | Incredible | Incredible | Incredible |
| | | | | | | | Rating Data Type | | | | | |
| | | | | | | | | | Methio- | | | |
| | | | | | | | Alanine | Valine | nine | Isoleucine | Leucine | Lysine |
| | | | | | | | Rating Unit | | | | | |
| Trt | Treatment | Form | | Rate | Other | Other | % | % | % | % | % | % |
| No. | Name | Conc | Rate | Unit | Rate | Rate Unit | of check | of check | of check | of check | of check | of check |
| 1 | Nicosulfuron*ACCENT | 75 | 0.66 | oz/a | 0.031 | lb ai/a | 1 | 0 | 0 | 0 | 0 | 1 |
| | Atrazine*AATREX | 4 | 16 | oz/a | 1 | pt/a | | | | | | |
| 2 | Mesotrione*CALLISTO | 4 | 3 | oz/a | 0.094 | lb ai/a | 4 | 7 | 2 | 6 | 6 | 12 |
| | Atrazine*AATREX | 4 | 16 | oz/a | 1 | pt/a | | | | | | |
| 3 | Nicosulfuron*ACCENT | 75 | 0.66 | oz/a | 0.031 | lb ai/a | 5 | 7 | 10 | 6 | 10 | 8 |
| | Mesotrione*CALLISTO | 4 | 3 | oz/a | 0.094 | lb ai/a | | | | | | |
| | Atrazine*AATREX | 4 | 16 | oz/a | 1 | pt/a | | | | | | |
| 4 | Tembotrione*LAUDIS | 3.5 | 3 | oz/a | 0.082 | lb ai/a | 7 | 8 | 8 | 6 | 9 | 10 |
| | Atrazine*AATREX | 4 | 16 | oz/a | 1 | pt/a | | | | | | |
| 5 | Topramezone*IMPACT | 2.8 | 0.75 | oz/a | 0.0164 | lb ai/a | 8 | 9 | 8 | 10 | 8 | 10 |
| | Atrazine*AATREX | 4 | 16 | oz/a | 1 | pt/a | | | | | | |
| 6 | Nicosulfuron*ACCENT Q | 54.5 | 0.9 | oz/a | 0.0307 | lb ai/a | 1 | 13 | 4 | 14 | 12 | 28 |
| | Atrazine*AATREX | 4 | 16 | oz/a | 1 | pt/a | | | | | | |
| 7 | Nicosulfuron*ACCENT Q | 54.5 | 0.9 | oz/a | 0.0307 | lb ai/a | 6 | −1 | 8 | 0 | 1 | 6 |
| | Topramezone*IMPACT | 2.8 | 0.75 | oz/a | 0.0164 | lb ai/a | | | | | | |
| | Atrazine*AATREX | 4 | 16 | oz/a | 1 | pt/a | | | | | | |
| 8 | Nicosulfuron*ACCENT Q | 54.5 | 0.9 | oz/a | 0.0307 | lb ai/a | 0 | 5 | 4 | 3 | 4 | 6 |
| | Mesotrione*CALLISTO | 4 | 3 | oz/a | 0.094 | lb ai/a | | | | | | |
| | Atrazine*AATREX | 4 | 16 | oz/a | 1 | pt/a | | | | | | |
| 9 | Atrazine*AATREX | 4 | 16 | oz/a | 1 | pt/a | 0 | 0 | 0 | 0 | 0 | 0 |

[symbol]

indicates data missing or illegible when filed

TABLE 3

| | | | | | | | Crop Name | | | | |
|---|---|---|---|---|---|---|---|---|---|---|---|
| | | | | | | | Sweet corn | Sweet corn | Sweet corn | Sweet corn | Sweet corn |
| | | | | | | | Crop Variety | | | | |
| | | | | | | | Incredible | Incredible | Incredible | Incredible | Incredible |
| | | | | | | | Rating Data Type | | | | |
| | | | | | | | calcium | phosphorous | magnesium | potassium | sodium |
| Trt | Treatment | Form | | Rate | Other | Other | Rating Unit | | | | |
| No. | Name | Conc | Rate | Unit | Rate | Rate Unit | % of check | % of check | % of check | % of check | % of check |
| 1 | Nicosulfuron*ACCENT | 75 | 0.66 | oz/a | 0.031 | lb ai/a | 0 | 6 | 14 | 1 | 21 |
| | Atrazine*AATREX | 4 | 16 | oz/a | 1 | pt/a | | | | | |
| 2 | Mesotrione*CALLISTO | 4 | 3 | oz/a | 0.094 | lb ai/a | 0 | 12 | 20 | 12 | 21 |
| | Atrazine*AATREX | 4 | 16 | oz/a | 1 | pt/a | | | | | |
| 3 | Nicosulfuron*ACCENT | 75 | 0.66 | oz/a | 0.031 | lb ai/a | 0 | 3 | 6 | 3 | 14 |
| | Mesotrione*CALLISTO | 4 | 3 | oz/a | 0.094 | lb ai/a | | | | | |
| | Atrazine*AATREX | 4 | 16 | oz/a | 1 | pt/a | | | | | |
| 4 | Tembotrione*LAUDIS | 3.5 | 3 | oz/a | 0.082 | lb ai/a | 0 | 5 | 14 | 3 | 7 |
| | Atrazine*AATREX | 4 | 16 | oz/a | 1 | pt/a | | | | | |
| 5 | Topramezone*IMPACT | 2.8 | 0.75 | oz/a | 0.0164 | lb ai/a | 0 | 8 | 13 | 6 | 29 |
| | Atrazine*AATREX | 4 | 16 | oz/a | 1 | pt/a | | | | | |
| 6 | Nicosulfuron*ACCENT Q | 54.5 | 0.9 | oz/a | 0.0307 | lb ai/a | 0 | 14 | 41 | 18 | 21 |
| | Atrazine*AATREX | 4 | 16 | oz/a | 1 | pt/a | | | | | |
| 7 | Nicosulfuron*ACCENT Q | 54.5 | 0.9 | oz/a | 0.0307 | lb ai/a | 0 | 8 | 27 | 5 | 36 |
| | Topramezone*IMPACT | 2.8 | 0.75 | oz/a | 0.0164 | lb ai/a | | | | | |
| | Atrazine*AATREX | 4 | 16 | oz/a | 1 | pt/a | | | | | |
| 8 | Nicosulfuron*ACCENT Q | 54.5 | 0.9 | oz/a | 0.0307 | lb ai/a | 100 | 8 | 20 | 3 | 0 |
| | Mesotrione*CALLISTO | 4 | 3 | oz/a | 0.094 | lb ai/a | | | | | |
| | Atrazine*AATREX | 4 | 16 | oz/a | 1 | pt/a | | | | | |
| 9 | Atrazine*AATREX | 4 | 16 | oz/a | 1 | pt/a | 0 | 0 | 0 | 0 | 0 |

| | | | | | | | Crop Name | | | |
|---|---|---|---|---|---|---|---|---|---|---|
| | | | | | | | Sweet corn | Sweet corn | Sweet corn | Sweet corn |
| | | | | | | | Crop Variety | | | |
| | | | | | | | Incredible | Incredible | Incredible | Incredible |
| | | | | | | | Rating Data Type | | | |
| | | | | | | | iron | manganese | zinc | copper |
| Trt | Treatment | Form | | Rate | Other | Other | Rating Unit | | | |
| No. | Name | Conc | Rate | Unit | Rate | Rate Unit | % of check | % of check | % of check | % of check |
| 1 | Nicosulfuron*ACCENT | 75 | 0.66 | oz/a | 0.031 | lb ai/a | 14 | 33 | 2 | 0 |
| | Atrazine*AATREX | 4 | 16 | oz/a | 1 | pt/a | | | | |
| 2 | Mesotrione*CALLISTO | 4 | 3 | oz/a | 0.094 | lb ai/a | 21 | 25 | 16 | 0 |
| | Atrazine*AATREX | 4 | 16 | oz/a | 1 | pt/a | | | | |
| 3 | Nicosulfuron*ACCENT | 75 | 0.66 | oz/a | 0.031 | lb ai/a | 3 | 8 | −5 | −25 |
| | Mesotrione*CALLISTO | 4 | 3 | oz/a | 0.094 | lb ai/a | | | | |
| | Atrazine*AATREX | 4 | 16 | oz/a | 1 | pt/a | | | | |
| 4 | Tembotrione*LAUDIS | 3.5 | 3 | oz/a | 0.082 | lb ai/a | 10 | 33 | 8 | 0 |
| | Atrazine*AATREX | 4 | 16 | oz/a | 1 | pt/a | | | | |
| 5 | Topramezone*IMPACT | 2.8 | 0.75 | oz/a | 0.0164 | lb ai/a | 23 | 38 | 4 | 0 |
| | Atrazine*AATREX | 4 | 16 | oz/a | 1 | pt/a | | | | |
| 6 | Nicosulfuron*ACCENT Q | 54.5 | 0.9 | oz/a | 0.0307 | lb ai/a | 31 | 42 | 51 | 0 |
| | Atrazine*AATREX | 4 | 16 | oz/a | 1 | pt/a | | | | |
| 7 | Nicosulfuron*ACCENT Q | 54.5 | 0.9 | oz/a | 0.0307 | lb ai/a | 67 | 75 | 14 | 0 |
| | Topramezone*IMPACT | 2.8 | 0.75 | oz/a | 0.0164 | lb ai/a | | | | |
| | Atrazine*AATREX | 4 | 16 | oz/a | 1 | pt/a | | | | |
| 8 | Nicosulfuron*ACCENT Q | 54.5 | 0.9 | oz/a | 0.0307 | lb ai/a | 26 | 38 | 9 | 0 |
| | Mesotrione*CALLISTO | 4 | 3 | oz/a | 0.094 | lb ai/a | | | | |
| | Atrazine*AATREX | 4 | 16 | oz/a | 1 | pt/a | | | | |
| 9 | Atrazine*AATREX | 4 | 16 | oz/a | 1 | pt/a | 0 | 0 | 0 | 0 |

TABLE 4

| | | | | | | | Crop Name | | | |
|---|---|---|---|---|---|---|---|---|---|---|
| | | | | | | | Sweet corn | Sweet corn | Sweet corn | Sweet corn |
| | | | | | | | Crop Variety | | | |
| | | | | | | | Incredible | Incredible | Incredible | Incredible |
| | | | | | | | Rating Data Type | | | |
| | | | | | | | tot fattyac | sat fattyac | mon fattyac | polyfattyac |
| Trt | Treatment | Form | | Rate | Other | Other | Rating Unit | | | |
| No. | Name | Conc | Rate | Unit | Rate | Rate Unit | % of check | % of check | % of check | % of check |
| 1 | Nicosulfuron*ACCENT | 75 | 0.66 | oz/a | 0.031 | lb ai/a | 13 | 12 | 15 | 12 |
| | Atrazine*AATREX | 4 | 16 | oz/a | 1 | pt/a | | | | |
| 2 | Mesotrione*CALLISTO | 4 | 3 | oz/a | 0.094 | lb ai/a | 11 | 7 | 9 | 12 |
| | Atrazine*AATREX | 4 | 16 | oz/a | 1 pt/a | | | | | |
| 3 | Nicosulfuron*ACCENT | 75 | 0.66 | oz/a | 0.031 | lb ai/a | 2 | 1 | 5 | 1 |
| | Mesotrione*CALLISTO | 4 | 3 | oz/a | 0.094 | lb ai/a | | | | |
| | Atrazine*AATREX | 4 | 16 | oz/a | 1 | pt/a | | | | |
| 4 | Tembotrione*LAUDIS | 3.5 | 3 | oz/a | 0.082 | lb ai/a | 11 | 10 | 16 | 8 |
| | Atrazine*AATREX | 4 | 16 | oz/a | 1 | pt/a | | | | |
| 5 | Topramezone*IMPACT | 2.8 | 0.75 | oz/a | 0.0164 | lb ai/a | 9 | 8 | 10 | 9 |
| | Atrazine*AATREX | 4 | 16 | oz/a | 1 | pt/a | | | | |
| 6 | Nicosulfuron*ACCENT Q | 54.5 | 0.9 | oz/a | 0.0307 | lb ai/a | 36 | 30 | 28 | 44 |
| | Atrazine*AATREX | 4 | 16 | oz/a | 1 | pt/a | | | | |
| 7 | Nicosulfuron*ACCENT Q | 54.5 | 0.9 | oz/a | 0.0307 | lb ai/a | 11 | 10 | 16 | 9 |
| | Topramezone*IMPACT | 2.8 | 0.75 | oz/a | 0.0164 | lb ai/a | | | | |
| | Atrazine*AATREX | 4 | 16 | oz/a | 1 | pt/a | | | | |
| 8 | Nicosulfuron*ACCENT Q | 54.5 | 0.9 | oz/a | 0.0307 | lb ai/a | 10 | 6 | 14 | 8 |
| | Mesotrione*CALLISTO | 4 | 3 | oz/a | 0.094 | lb ai/a | | | | |
| | Atrazine*AATREX | 4 | 16 | oz/a | 1 | pt/a | | | | |
| 9 | Atrazine*AATREX | 4 | 16 | oz/a | 1 | pt/a | 0 | 0 | 0 | 0 |

### EXAMPLE 2

A field study was conducted at Greenback, TN to investigate POST applications of several herbicide inhibitors of photosynthesis for sweet corn safety and for changes in carbohydrate content in the kernels of sweet corn among treatments. Treatments included bentazon at 1120 and 2240 g ai/ha, linuron at 280 and 560 g ai/ha, metribuzin at 105 and 210 g ai/ha, and bromoxynil at 560 and 1120 g ai/ha. All treatments included a PRE application of a common residual corn herbicide in order to maintain weed free conditions. Visual injury was rated on a percent scale from 0 to 100 where 0 equals no visual response and 100 equals complete plant death. Sweet corn was harvested from the center two rows of each four row plot and weighed in the husk. Eight ears of corn were harvested from each plot and were pooled over replicates for analysis of key carbohydrates which included the sugars sucrose, fructose, glucose, maltose, and raffanose.

Only bentazon at 1120 and 2240 g ai/ha and metribuzin at 105 g ai/ha caused less than 20% injury to sweet corn by 14 DAT, while all other treatments caused 22 to 47% injury. Metribuzin at 210 g ai/ha provided the greatest increases in carbohydrates with fructose, glucose, sucrose, maltose, and raffanose being increased 115%, 106%, 103%, 74%, and 59%, respectively compared to the untreated check. Similarly, bentazon at 560 or 1120 g ai/ha, linuron at 280 g ai/ha, and bromoxynil at 560 g ai/ha increased all of these sugary carbohydrates between 10 to 107% over the untreated check.

TABLE 5

| | | | | Sweet Corn Variety | | | |
|---|---|---|---|---|---|---|---|
| | | | | WH0809 | WH0809 | WH0809 | WH0809 |
| | | | | Rating Data Type | | | |
| | | | | Injury | Injury | Injury | YIELD |
| Trt | Treatment | | Rate | Rating Unit | | | |
| No. | Name | Rate | Unit | % | % | % | KG |
| 1 | BENTAZON | 1120 | g ai/ha | 5 | 17 | 0 | 10317 |
| | COC | 1 | % v/v | | | | |
| 2 | BENTAZON | 2240 | g ai/ha | 12 | 10 | 2 | 10565.6 |
| | COC | 1 | % v/v | | | | |
| 3 | LINURON | 280 | g ai/ha | 25 | 22 | 25 | 8062.6 |
| | COC | 1 | % v/v | | | | |
| 4 | LINURON | 560 | g ai/ha | 38 | 35 | 27 | 9227.9 |
| | COC | 1 | % v/v | | | | |
| 5 | METRIBUZIN | 105 | g ai/ha | 12 | 13 | 5 | 10715.4 |
| | COC | 1 | % v/v | | | | |
| 6 | METRIBUZIN | 210 | g ai/ha | 17 | 30 | 22 | 8967.5 |
| | COC | 1 | % v/v | | | | |
| 7 | BROMOXYNIL | 560 | g ai/ha | 37 | 35 | 27 | 9436.2 |
| | COC | 1 | % v/v | | | | |
| 8 | BROMOXYNIL | 1120 | g ai/ha | 45 | 47 | 35 | 9142.6 |
| | COC | 1 | % v/v | | | | |
| 9 | Treated check | | | 0 | 0 | 0 | 9758.4 |

TABLE 6

| | | | | Sweet corn variety | | | | |
|---|---|---|---|---|---|---|---|---|
| | | | | WH0809 | WH0809 | WH0809 | WH0809 | WH0809 |
| | | | | Part Rated | | | | |
| | | | | Sugar | Sugar | Sugar | Sugar | Sugar |
| | | | | content | content | content | content | content |
| | | | | Sugar Data Type | | | | |
| | | | | Fructose | Glucose | Sucrose | Maltose | Raffanose |
| Trt | | | Rate | Rating Unit | | | | |
| # | Treatment | Rate | Unit | mg/g dw | mg/g dw | mg/g dw | mg/g dw | mg/g dw |
| 1 | BENTAZON | 1120 | g ai/ha | 1.69 | 2.65 | 13.18 | 0.96 | 0.56 |
| | COC | 1 | % v/v | | | | | |
| 2 | BENTAZON | 2240 | g ai/ha | 1.78 | 3.41 | 18.67 | 1.26 | 0.84 |
| | COC | 1 | % v/v | | | | | |
| 3 | LINURON | 280 | g ai/ha | 1.86 | 3.22 | 12.02 | 0.95 | 0.64 |
| | COC | 1 | % v/v | | | | | |
| 4 | LINURON | 560 | g ai/ha | 1.39 | 2.33 | 13.30 | 0.59 | 0.48 |
| | COC | 1 | % v/v | | | | | |
| 5 | METRIBUZIN | 105 | g ai/ha | 1.04 | 1.79 | 9.35 | 0.49 | 0.36 |
| | COC | 1 | % v/v | | | | | |
| 6 | METRIBUZIN | 210 | g ai/ha | 2.24 | 3.93 | 21.79 | 1.06 | 0.81 |
| | COC | 1 | % v/v | | | | | |
| 7 | BROMOXYNIL | 560 | g ai/ha | 1.74 | 3.07 | 15.56 | 0.88 | 0.77 |
| | COC | 1 | % v/v | | | | | |
| 8 | BROMOXYNIL | 1120 | g ai/ha | 1.77 | 2.70 | 14.50 | 0.60 | 0.54 |
| | COC | 1 | % v/v | | | | | |
| 9 | Treated Check | — | — | 1.04 | 1.91 | 10.72 | 0.61 | 0.51 |

### EXAMPLE 3

Studies were conducted in a field in Knoxville, Tenn. to ascertain the growth, stress tolerance and nutritional quality differences following postemergence applications of herbicides mesotrione and atrazine, alone and in mixtures together, on the young sweet corn cultivars: ‘Merit’ (yellow-kernel sensitive genotype), ‘Temptation’ (bicolor tolerant genotype), and ‘Incredible’ (yellow-kernel moderately sensitive genotype).

Herbicide treatments were applied as follows: 1) untreated control; 2) mesotrione (meso) at 105 g ai/ha EPOST; 3) mesotrione at 105+atrazine at 560 g ai/ha EPOST; 4) atrazine at 560 g ai/ha EPOST; 5) mesotrione at 105 g ai/ha LPOST; 6) mesotrione at 105+atrazine at 560 g ai/ha LPOST; and 7) atrazine at 560 g ai/ha LPOST. Visual ratings of plant growth were taken during season, and kernels for carotenoids were measured at harvest.

In general, herbicide treatments enhanced carotenoid production in sweet corn, wherein EPOST applications of mesotrione at 105+atrazine at 560 g ai/ha yielded the greatest increase in nutrient production across varieties (Tables 7-8). In addition, sweet corn with different genetic compositions exhibited varying degrees of sensitivity to herbicide applications (Table 9).

TABLE 7
Effects of Herbicide Treatments on Kernel Carotenoids
of ‘Merit’ - Sensitive Genotype

| Treatment | Timing | Lutein | % change | Zeaxanthin | % change |
|---|---|---|---|---|---|
| Untreated | | 0.062 | | 0.509 | |
| Meso | EPOST | 0/881 | +7.8 | 0.570 | +12.0 |
| Meso + | EPOST | 0.706 | +11.7 | 0.583 | +12.7 |
| Atrazine | | | | | |
| Atrazine | EPOST | 0.692 | +9.5 | 0.564 | +10.8 |
| Meso | LPOST | 0.577 | +8.7 | 0.409 | −3.7 |
| Meso + | LPOST | 0.672 | +6.3 | 0.582 | +14.8 |
| Atrazine | | | | | |
| Atrazine | LPOST | 0.664 | +5.1 | 0.550 | +8.1 |

% change from untreated control

TABLE 8
Effects of Herbicide Treatments on Kernel Carotenoids
of ‘Incredible’ - Moderately sensitive Genotype

| Treatment | Timing | Lutein | % change | Zeaxanthin | % change |
|---|---|---|---|---|---|
| Untreated | | 0.328 | | 0.448 | |
| Meso | EPOST | 0.307 | −6.4 | 0.415 | −7.4 |
| Meso + | EPOST | 0.399 | +21.6 | 0.498 | +16.4 |
| Atrazine | | | | | |
| Atrazine | EPOST | 0.353 | +7.6 | 0.447 | −0.0 |
| Meso | LPOST | 0.343 | +4.6 | 0.454 | +1.3 |
| Meso + | LPOST | 0.340 | +3.7 | 0.436 | −2.8 |
| Atrazine | | | | | |
| Atrazine | LPOST | 0.347 | +6.0 | 0.454 | +1.3 |

% change from untreated control

TABLE 9
Effects of Herbicide Treatments of Visual Leaf Tissue Bleaching

| | | Leaf Tissue Visual Bleaching (%) | | | | | |
|---|---|---|---|---|---|---|---|
| | | ‘Merit’ | | | ‘Incredible’ | | |
| Treatment^{a} | Timing^{b} | 7 DAT | 14 DAT | 21 DAT | 7 DAT | 14 DAT | 21 DAT |
| Untreated | | 0 | 0 | 0 | 0 | 0 | 0 |
| Mesotrione | EPOST | 61 | 53 | 44 | 16 | 9 | 8 |
| Mesotrione + Atrazine | EPOST | 80 | 79 | 73 | 35 | 19 | 9 |
| Atrazine | EPOST | 3 | 2 | 0 | 13 | 13 | 0 |
| Untreated | | 0 | 0 | 0 | 0 | 0 | 0 |
| Mesotrione | LPOST | 34 | 29 | 9 | 11 | 9 | 1 |
| Mesotrione + Atrazine | LPOST | 39 | 33 | 13 | 11 | 10 | 2 |
| Atrazine | LPOST | 0 | 0 | 0 | 0 | 0 | 0 |

### EXAMPLE 4

Greenhouse and laboratory studies were conducted to ascertain the tolerance and nutritional quality differences following postemergence applications of the insecticide chlorpyrifos, the fungicide chlorothalonil, the PGRs mepiquat-chloride and paclobutrazol, and the safeners napthalic anhydride and isoxadifen-ethyl on kale ‘Red Russian’ and basil ‘Genovese’. Visual injury was rated based on general phytotoxicity, chlorosis, and visual stunting and each measurement was based on a percent scale from 0 to 100 where 0 equals no visual response and 100 equals complete plant death. Kale and basil were both harvested approximately 4 weeks after treatments and plants were pooled over replicates for content analysis of crude protein, crude amino acid content, acid detergent fiber, neutral detergent fiber, total fatty acids, and/or key mineral content (including Na, Ca, P, Mg, K, Fe, Mn, Zn, and Cu). In addition, the amounts of key individual amino acids (aspartic acid, threonine, glutamic acid, proline, glycine, cysteine, valine, leucine, isoleucine, methionine, and lysine) and composite classifications of fatty acid (saturated fatty acids, monounsaturated fatty acids, polyunsaturated fatty acids) were also analyzed from these sub-samples for kale (Tables 10-14). Phytonutrient amounts were converted to a percentage of the treated check to highlight the differences observed due to each herbicide(s) with or without safener application.

All treatments injured kale and basil between 0 to 28%. All pesticides (fungicide, insecticide, PGRs, and safeners) increased the level of at least one mineral element in kale and/or basil. The insecticide chlorpyrifos at 1120 g ai/ha increased all mineral elements by 4 to 108%, while the plant growth regulator paclobutrazol increased total protein, total amino acids, individual amino acids, and all mineral elements (except iron) in kale by 2 to 28% when applied at 9.7 g ai/ha. The safener isoxadifen increased mineral elements by 6 to 118% (except copper) when applied at 16 g ai/ha in basil. Similarly, the safener napthalic anhydride increase mineral elements by 2 to 47% (except copper and potassium) when applied at 264 g ai/ha in basil. All treatments increased soluble protein and iron in basil by 4 to 223% over the untreated check.

### EXAMPLE 5

Greenhouse and laboratory studies were initiated with ‘Riviera’ bermudagrass (Cynodon dactylon (L.) Pers.) treated postemergence with mesotrione at 0.28 kg×ha⁻¹ and sampled for tissue pigment concentrations at 0, 3, 7, 14, 21, 28 and 35 days after treatment (DAT; Table 16). Visual tissue whitening in mesotrione treated plants reached a maximum of 38% by 14 DAT; however, re-greening of discolored tissue was observed by 21 DAT. Phytoene was only detected in mesotrione treated plants at 3, 7, and 14 DAT. Pigments in treated plants decreased with initial tissue whitening; however, most recovered to untreated levels by 21 DAT. At 35 DAT, lutein, β-carotene, and zeaxanthin in mesotrione treated plants had accumulated to levels exceeding untreated control plants. Study results demonstrate initial decreases in pigment concentrations induced by mesotrione eventually lead to higher concentrations of carotenoid pigments in bermudagrass leaf tissues at 28 to 35 DAT. Due to the antioxidant activity of these carotenoid pigments, these increases should improve bermudagrass stress tolerance (Kopsell et al., 2010).

### EXAMPLE 6

Greenhouse and laboratory studies were conducted to ascertain the tolerance and potential sugar enhancement from postemergence applications of the cellulose biosynthesis inhibiting herbicide isoxaben at 1120 g ai/ha plus 1% v/v crop oil concentrate as an adjuvant applied postemergence on switchgrass “Alamo”. Switchgrass was harvested 8 weeks after application and three replicates were analyzed compared with an untreated check. Sugar content was assessed on a percentage of the fresh weight and values were converted to a percentage of values derived from the untreated control. Isoxaben increased fructose by 44%, glucose by 33%, and total sugars by 38% in comparison to the untreated check. Switchgrass is currently under evaluations as a biofuels crop whereby enhanced sugar output per acre is desired.

TABLE 10
Basil treated with insecticide, fungicide, PGRs and safeners

| | | | | | | | | | | | | Yield |
|---|---|---|---|---|---|---|---|---|---|---|---|---|
| | | | Phytotoxicity | | | | | | | | | Fresh |
| | | | General | Chlorosis | Stunting | General | Chlorosis | Stunting | General | Chlorosis | Stunting | weight |
| | | | % | % | % | % | % | % | % | % | % | grams |
| Treatment | Rate | Unit | 7 DA-A | 7 DA-A | 7 DA-A | 14 DA-A | 14 DA-A | 14 DA-A | 28 DA-A | 28 DA-A | 28 DA-A | DA-A |
| chlropyrifos | 1120 | g ai/ha | 2 | 8 | 5 | 7 | 3 | 10 | 10 | 5 | 8 | 24.6 |
| NIS | 0.25 | % v/v | | | | | | | | | | |
| chlropyrifos | 3370 | g ai/ha | 2 | 10 | 13 | 2 | 8 | 10 | 2 | 7 | 12 | 32.9 |
| NIS | 0.25 | % v/v | | | | | | | | | | |
| chlorothalonil | 2630 | g ai/ha | 5 | 5 | 5 | 0 | 5 | 8 | 10 | 5 | 7 | 27.5 |
| NIS | 0.25 | % v/v | | | | | | | | | | |
| chlorothalonil | 7900 | g ai/ha | 3 | 15 | 5 | 2 | 5 | 7 | 3 | 10 | 10 | 25.9 |
| NIS | 0.25 | % v/v | | | | | | | | | | |
| paclobutrazol | 3.23 | g ai/ha | 3 | 8 | 3 | 0 | 5 | 7 | 5 | 3 | 8 | 22.4 |
| NIS | 0.25 | % v/v | | | | | | | | | | |
| paclobutrazol | 9.7 | g ai/ha | 3 | 5 | 2 | 0 | 7 | 5 | 2 | 7 | 2 | 24.4 |
| NIS | 0.25 | % v/v | | | | | | | | | | |
| mepiquat-chloride | 12.3 | g ai/ha | 2 | 3 | 3 | 5 | 7 | 3 | 2 | 2 | 2 | 36.9 |
| NIS | 0.25 | % v/v | | | | | | | | | | |
| mepiquat-chloride | 37 | g ai/ha | 2 | 5 | 5 | 5 | 2 | 0 | 0 | 2 | 0 | 35.6 |
| NIS | 0.25 | % v/v | | | | | | | | | | |
| Isoxadifen | 8 | g ai/ha | 3 | 8 | 2 | 2 | 5 | 5 | 2 | 2 | 5 | 34.2 |
| NIS | 0.25 | % v/v | | | | | | | | | | |
| Isoxadifen | 16 | g ai/ha | 7 | 7 | 7 | 8 | 8 | 10 | 7 | 0 | 8 | 36.9 |
| NIS | 0.25 | % v/v | | | | | | | | | | |
| Napthalic | 264 | g ai/ha | 5 | 0 | 0 | 7 | 3 | 0 | 5 | 2 | 0 | 41.5 |
| anhydride | | | | | | | | | | | | |
| NIS | 0.25 | % v/v | | | | | | | | | | |
| Napthalic | 527 | g ai/ha | 7 | 2 | 5 | 10 | 2 | 7 | 3 | 2 | 7 | 24.5 |
| anhydride | | | | | | | | | | | | |
| NIS | 0.25 | % v/v | | | | | | | | | | |
| check | | | 0 | 0 | 0 | 0 | 0 | 0 | 0 | 0 | 0 | 31.8 |

TABLE 11
Basil treated with insecticide, fungicide, PGRs and safeners

| | | | Percent Increase Over the Untreated Check (on a % dry matter basis) | | | | | | |
|---|---|---|---|---|---|---|---|---|---|
| | | | ADF % | NDF % | Calcium % | | | | Sodium % |
| | | | check | check | check | Phosphorous | Magnesium | Potassium | check |
| | | | 28 | 28 | 28 | % check | % check | % check | 28 |
| Treatment | Rate | Unit | DA-A | DA-A | DA-A | 28 DA-A | 28 DA-A | 28 DA-A | DA-A |
| chlropyrifos | 1120 | g ai/ha/m ch | −12 | −8 | 7 | 11 | 12 | 12 | 83 |
| NIS | 0.25 | % v/v | | | | | | | |
| chlroprifos | 3370 | g ai/ha/m ch | 10 | 9 | 3 | 50 | 21 | 27 | 25 |
| NIS | 0.25 | % v/v | | | | | | | |
| chlorothalonil | 2630 | g ai/ha/m ch | 1 | 6 | 0 | 9 | 3 | 4 | 8 |
| NIS | 0.25 | % v/v | | | | | | | |
| chlorothalonil | 7900 | g ai/ha/m ch | 1 | 12 | −15 | 0 | −6 | 7 | 50 |
| NIS | 0.25 | % v/v | | | | | | | |
| paclobutrazol | 3.23 | g ai/ha/m ch | 7 | 7 | −2 | 2 | −8 | −1 | −25 |
| NIS | 0.25 | % v/v | | | | | | | |
| paclobutrazol | 9.7 | g ai/ha/m ch | −12 | −8 | −18 | 0 | −9 | −5 | −17 |
| NIS | 0.25 | % v/v | | | | | | | |
| mepiquat- | 12.3 | g ai/ha/m ch | 7 | −8 | 2 | 13 | 0 | −4 | −8 |
| chloride | | | | | | | | | |
| NIS | 0.25 | % v/v | | | | | | | |
| mepiquat- | 37 | g ai/ha/m ch | −9 | −11 | 2 | 26 | 16 | −3 | 8 |
| chloride | | | | | | | | | |
| NIS | 0.25 | % v/v | | | | | | | |
| Isoxadifen | 8 | g ai/ha | 6 | 1 | −2 | 17 | 9 | 17 | 27 |
| NIS | 0.25 | % v/v | | | | | | | |
| Isoxadifen | 16 | g ai/ha | 13 | 0 | 6 | 28 | 12 | 16 | 17 |
| NIS | 0.25 | % v/v | | | | | | | |
| Napthalic | 264 | g ai/ha | 13 | 2 | 2 | 22 | 12 | −4 | 33 |
| anhydride | | | | | | | | | |
| NIS | 0.25 | % v/v | | | | | | | |
| Napthalic | 527 | g ai/ha | 4 | 1 | −8 | −9 | −6 | −23 | −8 |
| anhydride | | | | | | | | | |
| NIS | 0.25 | % v/v | | | | | | | |
| check | | | 0 | 0 | 0 | 0 | 0 | 0 | 0 |

| | | | Percent Increase Over the Untreated Check (on a % dry matter basis) | | | | | |
|---|---|---|---|---|---|---|---|---|
| | | | | | | | | Soluble |
| | | | Iron | | | Copper | Protein % | protein % |
| | | | % check | Manganese | Zinc | % check | check | check |
| | | | 28 | % check | % check | 28 | 28 | 28 |
| Treatment | Rate | Unit | DA-A | 28 DA-A | 28 DA-A | DA-A | DA-A | DA-A |
| chlropyrifos | 1120 | g ai/ha/m ch | 108 | 28 | 33 | 8 | 20 | 196 |
| NIS | 0.25 | % v/v | | | | | | |
| chlroprifos | 3370 | g ai/ha/m ch | 57 | 12 | 46 | 8 | 32 | 42 |
| NIS | 0.25 | % v/v | | | | | | |
| chlorothalonil | 2630 | g ai/ha/m ch | 37 | 43 | 38 | −17 | 3 | 223 |
| NIS | 0.25 | % v/v | | | | | | |
| chlorothalonil | 7900 | g ai/ha/m ch | 25 | −8 | −6 | −17 | 3 | 42 |
| NIS | 0.25 | % v/v | | | | | | |
| paclobutrazol | 3.23 | g ai/ha/m ch | 63 | 26 | 28 | 8 | 6 | 50 |
| NIS | 0.25 | % v/v | | | | | | |
| paclobutrazol | 9.7 | g ai/ha/m ch | 4 | −12 | −8 | −25 | −9 | 12 |
| NIS | 0.25 | % v/v | | | | | | |
| mepiquat- | 12.3 | g ai/ha/m ch | 14 | 61 | 34 | 0 | 13 | 89 |
| chloride | | | | | | | | |
| NIS | 0.25 | % v/v | | | | | | |
| mepiquat- | 37 | g ai/ha/m ch | 6 | 61 | 53 | −8 | 7 | 27 |
| chloride | | | | | | | | |
| NIS | 0.25 | % v/v | | | | | | |
| Isoxadifen | 8 | g ai/ha | 25 | 10 | 38 | 0 | 14 | 12 |
| NIS | 0.25 | % v/v | | | | | | |
| Isoxadifen | 16 | g ai/ha | 27 | 118 | 34 | 0 | 22 | 4 |
| NIS | 0.25 | % v/v | | | | | | |
| Napthalic | 264 | g ai/ha | 14 | 37 | 47 | 0 | 32 | 4 |
| anhydride | | | | | | | | |
| NIS | 0.25 | % v/v | | | | | | |
| Napthalic | 527 | g ai/ha | 0 | −16 | −13 | −17 | 3 | 31 |
| anhydride | | | | | | | | |
| NIS | 0.25 | % v/v | | | | | | |
| check | | | 0 | 0 | 0 | 0 | 0 | 0 |

TABLE 12
Kale treated with insecticide, fungicide, and PGRs

| | | | | | | | | | | | | Yield |
|---|---|---|---|---|---|---|---|---|---|---|---|---|
| | | | | | | | | | | | | Fresh |
| | | | Phytotoxicity | | | | | | | | | weight |
| | | | General | Chlorosis | Stunting | General | Chlorosis | Stunting | General | Chlorosis | Stunting | grams |
| | | | % | % | % | % | % | % | % | % | % | 28 |
| Treatment | Rate | Unit | 7 DA-A | 7 DA-A | 7 DA-A | 14 DA-A | 14 DA-A | 14 DA-A | 28 DA-A | 28 DA-A | 28 DA-A | DA-A |
| chlropyrifos | 1120 | g ai/ha/m | 14 | 1 | 9 | 4 | 15 | 9 | 4 | 6 | 8 | 108.2 |
| | | ch | | | | | | | | | | |
| NIS | 0.25 | % v/v | | | | | | | | | | |
| chlropyrifos | 3370 | g ai/ha/m | 13 | 0 | 6 | 5 | 13 | 10 | 3 | 3 | 6 | 110.1 |
| | | ch | | | | | | | | | | |
| NIS | 0.25 | % v/v | | | | | | | | | | |
| chlorothalonil | 2630 | g ai/ha/m | 15 | 0 | 0 | 0 | 10 | 5 | 5 | 3 | 6 | 102.5 |
| | | ch | | | | | | | | | | |
| NIS | 0.25 | % v/v | | | | | | | | | | |
| chlorothalonil | 7900 | g ai/ha/m | 9 | 0 | 1 | 1 | 6 | 5 | 3 | 0 | 8 | 107.5 |
| | | ch | | | | | | | | | | |
| NIS | 0.25 | % v/v | | | | | | | | | | |
| paclobutrazol | 3.23 | g ai/ha/m | 10 | 1 | 6 | 3 | 10 | 19 | 3 | 3 | 21 | 107.2 |
| | | ch | | | | | | | | | | |
| NIS | 0.25 | % v/v | | | | | | | | | | |
| paclobutrazol | 9.7 | g ai/ha/m | 9 | 0 | 11 | 6 | 16 | 28 | 4 | 0 | 19 | 108.3 |
| | | ch | | | | | | | | | | |
| NIS | 0.25 | % v/v | | | | | | | | | | |
| mepiquat- | 12.3 | g ai/ha/m | 9 | 0 | 4 | 1 | 15 | 16 | 1 | 6 | 9 | 101.8 |
| chloride | | ch | | | | | | | | | | |
| NIS | 0.25 | % v/v | | | | | | | | | | |
| mepiquat- | 37 | g ai/ha/m | 10 | 0 | 1 | 0 | 4 | 3 | 3 | 3 | 10 | 101.2 |
| chloride | | ch | | | | | | | | | | |
| NIS | 0.25 | % v/v | | | | | | | | | | |
| Untreated | | | 0 | 0 | 0 | 0 | 0 | 0 | 0 | 0 | 0 | 106.9 |
| Check | | | | | | | | | | | | |

TABLE 13
Kale treated with insecticide, fungicide, and PGRs

| | | | Percent Increase over the Untreated Check (on a % dry matter basis) | | | | | | | |
|---|---|---|---|---|---|---|---|---|---|---|
| | | | Aspartic | | | | | | | |
| | | | acid % | | Glutamic | Proline % | Glycine % | Alanine % | | Valine % |
| | | | check | Threonine | acid | check | check | check | Cysteine | check |
| | | | 28 | % check | % check | 28 | 28 | 28 | % check | 28 |
| Treatment | Rate | Unit | DA-A | 28 DA-A | 28 DA-A | DA-A | DA-A | DA-A | 28 DA-A | DA-A |
| chlropyrifos | ### | g ai/ha/m | 0 | −2 | 5 | −2 | −4 | −1 | 0 | 1 |
| | | ch | | | | | | | | |
| NIS | 0.3 | % v/v | | | | | | | | |
| chlropyrifos | ### | g ai/ha/m | 7 | 4 | 12 | 9 | 3 | 6 | 6 | 7 |
| | | ch | | | | | | | | |
| NIS | 0.3 | % v/v | | | | | | | | |
| chlorothalonil | ### | g ai/ha/m | 2 | 3 | 5 | 6 | 0 | 3 | −6 | 4 |
| | | ch | | | | | | | | |
| NIS | 0.3 | % v/v | | | | | | | | |
| chlorothalonil | ### | g ai/ha/m | −4 | −1 | −5 | −3 | −7 | −5 | −6 | −3 |
| | | ch | | | | | | | | |
| NIS | 0.3 | % v/v | | | | | | | | |
| paclobutrazol | 3.2 | g ai/ha/m | −9 | −9 | −5 | −4 | −12 | −9 | −6 | −6 |
| | | ch | | | | | | | | |
| NIS | 0.3 | % v/v | | | | | | | | |
| paclobutrazol | 9.7 | g ai/ha/m | 12 | 4 | 16 | 7 | 3 | 4 | 6 | 6 |
| | | ch | | | | | | | | |
| NIS | 0.3 | % v/v | | | | | | | | |
| mepiquat- | 12 | g ai/ha/m | 6 | 6 | 11 | 8 | 4 | 5 | 6 | 5 |
| chloride | | ch | | | | | | | | |
| NIS | 0.3 | % v/v | | | | | | | | |
| mepiquat- | 37 | g ai/ha/m | −2 | −1 | −2 | 2 | −7 | −4 | −6 | −2 |
| chloride | | ch | | | | | | | | |
| NIS | 0.3 | % v/v | | | | | | | | |
| Untreated | | | 0 | 0 | 0 | 0 | 0 | 0 | 0 | 0 |
| Check | | | | | | | | | | |

| | | | Percent Increase over the Untreated Check (on a % dry matter basis) | | | | | |
|---|---|---|---|---|---|---|---|---|
| | | | | | | | Total | Total |
| | | | | Iso- | | | Amino | Protein % |
| | | | Methionine | leucine | Leucine | Lysine | acids | check |
| | | | % check | % check | % check | % check | % check | 28 |
| Treatment | Rate | Unit | 28 DA-A | 28 DA-A | 28 DA-A | 28 DA-A | 28 DA-A | DA-A |
| chlropyrifos | ### | g ai/ha/m | 2 | 0 | 0 | 6 | 1 | 3 |
| | | ch | | | | | | |
| NIS | 0.3 | % v/v | | | | | | |
| chlropyrifos | ### | g ai/ha/m | 0 | 6 | 6 | 11 | 7 | 5 |
| | | ch | | | | | | |
| NIS | 0.3 | % v/v | | | | | | |
| chlorothalonil | ### | g ai/ha/m | 2 | 4 | 4 | 5 | 3 | 1 |
| | | ch | | | | | | |
| NIS | 0.3 | % v/v | | | | | | |
| chlorothalonil | ### | g ai/ha/m | −2 | −2 | −1 | −3 | −4 | −3 |
| | | ch | | | | | | |
| NIS | 0.3 | % v/v | | | | | | |
| paclobutrazol | 3.2 | g ai/ha/m | −13 | −6 | −7 | −8 | −8 | 0 |
| | | ch | | | | | | |
| NIS | 0.3 | % v/v | | | | | | |
| paclobutrazol | 9.7 | g ai/ha/m | 2 | 6 | 5 | 14 | 9 | 13 |
| | | ch | | | | | | |
| NIS | 0.3 | % v/v | | | | | | |
| mepiquat- | 12 | g ai/ha/m | 4 | 6 | 4 | 6 | 6 | 4 |
| chloride | | ch | | | | | | |
| NIS | 0.3 | % v/v | | | | | | |
| mepiquat- | 37 | g ai/ha/m | −4 | −3 | −4 | −1 | −2 | 0 |
| chloride | | ch | | | | | | |
| NIS | 0.3 | % v/v | | | | | | |
| Untreated | | | 0 | 0 | 0 | 0 | 0 | 0 |
| Check | | | | | | | | |

TABLE 14
Kale treated with insecticide, fungicide, PGRs and safeners

| | | | Percent Increase Over the Untreated Check (on a % dry matter basis) | | | | | | | | | | |
|---|---|---|---|---|---|---|---|---|---|---|---|---|---|
| | | | | | | Phos- | Mag- | | | | | | |
| | | | ADF | NDF | Calcium | phorous | nesium | | Sodium | Iron | | Zinc | Copper |
| | | | % | % | % | % | % | | % | % | | % | % |
| | | | check | check | check | check | check | Potassium | check | check | Manganese | check | check |
| | | | 28 | 28 | 28 | 28 | 28 | % check | 28 | 28 | % check | 28 | 28 |
| Treatment | Rate | Unit | DA-A | DA-A | DA-A | DA-A | DA-A | 28 DA-A | DA-A | DA-A | 28 DA-A | DA-A | DA-A |
| chlropyrifos | 1120 | g ai/ha/m | 1 | −8 | 18 | 9 | 11 | 4 | 13 | 33 | 39 | 18 | 50 |
| | | ch | | | | | | | | | | | |
| NIS | 0.25 | % v/v | | | | | | | | | | | |
| chlropyrifos | 3370 | g ai/ha/m | 9 | 1 | 0 | 7 | 3 | 1 | 45 | 3 | 16 | 10 | 28 |
| | | ch | | | | | | | | | | | |
| NIS | 0.25 | % v/v | | | | | | | | | | | |
| chlorothalonil | 2630 | g ai/ha/m | 9 | −8 | −6 | 0 | −3 | −9 | 4 | −1 | 3 | −4 | 6 |
| | | ch | | | | | | | | | | | |
| NIS | 0.25 | % v/v | | | | | | | | | | | |
| chlorothalonil | 7900 | g ai/ha/m | 0 | −5 | −1 | −3 | −3 | −3 | 23 | −3 | 4 | −3 | 6 |
| | | ch | | | | | | | | | | | |
| NIS | 0.25 | % v/v | | | | | | | | | | | |
| paclobutrazol | 3.23 | g ai/ha/m | 11 | −3 | 11 | 6 | 8 | 3 | −1 | −3 | 10 | 11 | 12 |
| | | ch | | | | | | | | | | | |
| NIS | 0.25 | % v/v | | | | | | | | | | | |
| paclobutrazol | 9.7 | g ai/ha/m | −1 | −8 | 22 | 12 | 11 | 15 | 11 | −5 | 20 | 13 | 28 |
| | | ch | | | | | | | | | | | |
| NIS | 0.25 | % v/v | | | | | | | | | | | |
| mepiquat- | 12.3 | g ai/ha/m | 7 | −2 | −4 | 1 | −3 | −5 | 4 | −5 | −2 | −5 | 0 |
| chloride | | ch | | | | | | | | | | | |
| NIS | 0.25 | % v/v | | | | | | | | | | | |
| mepiquat- | 37 | g ai/ha/m | −2 | −7 | −3 | 1 | 0 | −1 | 4 | −4 | −7 | −10 | −6 |
| chloride | | ch | | | | | | | | | | | |
| NIS | 0.25 | % v/v | | | | | | | | | | | |
| Untreated | | | 0 | 0 | 0 | 0 | 0 | 0 | 0 | 0 | 0 | 0 | 0 |
| Check | | | | | | | | | | | | | |

TABLE 15
Sugar and key antioxidant content of corn
Percent increase in levels of key sugars and antioxidants for sweet corn variety ‘Incredible’
following applications of carotenoid and amino acid biosynthesis inhibitors
applied alone and in mixtures with the Photosystem II inhibitors atrazine.

| | | Sugars | | | | | Antioxidants | | | |
|---|---|---|---|---|---|---|---|---|---|---|
| | | Total | | | | | | | | Gamma- |
| Herbicide | Rate | Sugars | Fructose | Glucose | Maltose | Sucrose | Lutein | Zeaxanthin | antheraxanthin | tocopherol |
| treatment^{a} | g ai/ha | % | | | | | | | | |
| Nicosulfuron | 35 | 16 | 48 | 35 | 23 | −4 | 9 | 9 | 15 | 1 |
| Mesotrione | 105 | −4 | 18 | 19 | 15 | −32 | 17 | 1 | 14 | 5 |
| Topramezone | 18 | 10 | 63 | 40 | 8 | −18 | 0 | −8 | 23 | 8 |
| Nicosulfuron + | 35 + 13 | 10 | 68 | 43 | −15 | −15 | 0 | −2 | 19 | −3 |
| Isoxadifen-ethyl | | | | | | | | | | |
| Nicosulfuron + | 35 + 18 + 13 | 11 | 63 | 42 | 8 | −21 | 2 | −5 | 49 | −10 |
| Topramezone + | | | | | | | | | | |
| Isoxadifen-ethyl | | | | | | | | | | |
| Nicosulfuron + | 35 + 105 + 13 | 9 | 31 | 23 | 4 | 2 | 7 | −1 | 21 | 0 |
| Mesotrione + | | | | | | | | | | |
| Isoxadifen-ethyl | | | | | | | | | | |
| Treated check | — | 0 | 0 | 0 | 0 | 0 | 0 | 0 | 0 | 0 |

^{a}All treatments included the treated check included a postemergence treatment of atrazine at 560 g ai/ha + 1% v/v crop oil concentrate.

TABLE 16
Leaf blade carotenoid pigments (mg × 100 g⁻¹ Fresh Weight) in ‘Riviera’
bermudagrass (Cynodon dactylon (L.) Pers.) treated with mesotrione
at 0.28 kg ai×ha⁻¹ and sampled at 0, 3, 7, 14, 21, 28 and 35 days
after mesotrione treatment application. Means pooled from two experimental runs.
Data adapted from Kopsell et al., 2010.

| Days after | Blade tissue pigments (mg/100 g fresh weight) | | | | | |
|---|---|---|---|---|---|---|
| Mesotrione | Total | | | Beta- | | |
| treatment | Carotenoids | Phytoene | Lutein | carotene | Zeaxanthin | Violaxanthin |
| 0 | 57.5 | 0.0 | 23.6 | 17.2 | 3.2 | 6.9 |
| 3 | 42.9 | 37.5 | 14.8 | 11.6 | 3.1 | 1.6 |
| 7 | 35.6 | 38.0 | 14.1 | 7.4 | 3.3 | 1.6 |
| 14 | 47.2 | 11.7 | 16.4 | 7.6 | 3.8 | 3.5 |
| 21 | 60.1 | 0.0 | 21.2 | 11.4 | 4.7 | 4.0 |
| 28 | 77.2 | 0.0 | 32.1 | 18.9 | 5.7 | 2.8 |
| 35 | 92.7 | 0.0 | 37.1 | 21.2 | 6.4 | 6.8 |

Kopsell, D. A., J. T. Brosnan, G. R. Armel, and J. S. McElroy. 2010, Increases in bermudagrass (Cynodon dactylon (L.) Pers.) tissue pigments during post-application recovery from mesotrione. HortScience 45(10): In Press.

We claim:

1. A method of increasing carbohydrate levels in a plant comprising applying a composition comprising one or more herbicides, defoliants/desiccants, insecticides, nematicides, miticides, fungicides, antibiotics, plant growth regulators, or any combination thereof to a plant, wherein said one or more herbicides, defoliants/desiccants, insecticides, nematicides, miticides, fungicides, antibiotics, plant growth regulators are applied alone or in combination with a safener other pesticide or agrochemical in an amount sufficient to increase carbohydrate levels in said plant.

2. The method according to claim 1, wherein said one or more herbicides, defoliants/desiccants, insecticides, nematicides, miticides, fungicides, antibiotics, plant growth regulators, has inhibit, activate, modulate, uncouple, up-regulate, mimic, disrupt, or otherwise modify: protoporphoryinogen IX oxidase (Protox), carotenoid biosynthesis, phytoene desaturase, deoxy-D-xylulose-5-phosphate reductoisomerase (DOXP reductoisomerase), p-hydroxyphenylpyruvate dioxygenase (IIPPD), zeta-carotene desaturase (ZDS), 1-deoxy-D-xylulose-5-phosphate synthatase (DOXP synthase), lycopene cyclase (LC), and phytoene desaturase (PDS), Photosystem I in photosynthesis, Photosystem II in photosynthesis acetolactate synthase, 5-enolpyruvylshikimate-3-phosphate synthase, glutamine synthetase microtubule organization or assembly in mitosis, formation of very long chain fatty acids, cellulose biosynthesis, auxin transport, internal auxins, nucleic acid synthesis; mitosis and/or cellular division; cellular respiration; amino acid and/or protein synthesis; lipids and/or membrane synthesis, cellular signal transduction, sterol biosynthesis, glucan synthesis, host-pathogen interactions, acetylcholinesterase, GABA-gated chloride channel antagonists, sodium channel modulators, nicotinic acetylcholine receptor agonists, nicotinic acetylcholine receptor allosteric activators, chloride channel activators, mimic juvenile hormones, Homopteran feeding, mite growth, activities of insect midgut membranes, mitochondrial ATP synthase, oxidative phosphorylation via proton gradient, nicotinic acetylcholine receptor channel blockers, chitin biosynthesis (type 1), molting disruptors (Dipteran), ecdysone receptor agonists, octopamine receptor agonists, mitochondrial complex III electron transport and mitochondrial complex I electron transport, voltage-dependent sodium channels, acetyl CoA carboxylase, mitochondrial complex IV electron transport and mitochondrial complex II electron transport, ryanodine receptors, gibberellic acid production, ethylene production, shoot elongation, cell division and cell elongation, ripening, overall plant growth and development.

3. The method according to claim 1, wherein said herbicides, defoliants/desiccants, insecticides, nematicides, miticides, fungicides, antibiotics, plant growth regulators, is selected from a group consisting of ametryn, bromacil, desmedipham, phenmedipham, hexazinone, atrazine, pyrazon, simazine, metribuzin, prometryn, prometon, amicarbazone, terbacil, bentazon, bromoxynil, ioxynil, pyridate, propanil, diuron, linuron, siduron, fluometuron, tebuthiuron, paraquat, diquat, sorgoleone, fischerellin A, juglone, anthroquinone, capsaicin, imazethapyr, imazapyr, imazaquin, imazamox imazamethabenz, imazapic, byspyribac, pyrithiobac, flucarbazone, thiencarbazone, propoxycarbazone, amidosulfuron, azimsulfuron, bensulfuron, chlorimuron, chlorsulfuron, ethoxysulfuron, flazasulfuron, flupyrsulfuron, foramsulfuron, halosulfuron, imazosulfuron, iodosulfuron, mesosulfuron, nicosulfuron, primisulfuron, rimsulfuron, sulfosulfuron, sulfometuron, thifensulfuron, tribenuron, trifloxysulfuron, triflusulfuron, cloransulam, florasulam, flumetsuam, penoxsulam, pyroxsulam, diclosulam, glyphosate, gliotoxin, bialaphos, fosmidomycin, mesotrione, sulcotrione, tembotrione, isoxaflutole, bicyclopyrone, pyrasulfatole, isoxachlortole, topramezone, benzofenap, pyrazoxyfen, DFPC, clomazone, norflurazon, flurochloridone, diflufenican, fluridone, picolinafen, flurtamone, beflubutamid, amitrole, CPTA acifluorfen, lactofen, oxyfluorfen, fomesafen, carfentrazone, sulfentrazone, azafenidin oxadiazon, oxadiargyl, cinidon, flumioxazin, flumiclorac, pyraflufen, fluazolate, saflufenacil, butafenacil, fluthiacet, leptospermone, pendimethalin, trifluralin, oryzalin, prodiamine, benefin, ethalfluralin, DCPA, butamiphos, amiprophos, pronamide, tebutam, carbetamide, propham, chlorpropham, clethodim, sethoxydim, tralkoxydim, fluazifop, fenoxaprop, quizalofop, diclofop, haloxyfop, pinoxaden, EPTC, pebulate, vernolate, molinate, triallate, butylate, bensulide, ethofumesate, benfuresate, TCA, dalapon, flupropanate, metazachlor, metolachlor, acetochlor, dimethenamid, pretilachlor, propachlor, alachlor, diphenamid, napropamide, naproanilide, flufenacet, mefenacet, fentrazamide, anilofos, piperophos, cafenstrole, indanofan, pyroxasulfone, dichlobenil, chlorthiamid, isoxaben, flupoxam, indaziflam, diflufenzopyr, naptalam, picloram, fluroxypyr, clopyralid, aminopyralid, triclopyr, aminocyclopyrachlor, 2,4-D, 2,4-DB, 2,4,5-T, dichlorprop, mecoprop, MCPA, MCPB, MCPP, dicamba, tricamba, chloramben, quinclorac, quinmerac, ailanthone, catechin, dimethipin, endothall, pelargonic acid, sodium chlorate, thiadiazuron, tribufos metalaxyl, mefenoxam, octhilinone, carbendazim, thisbendazole, zoxamide, flutolanil, carboxin, oxycarboxin, boscalid, azoxystrobin, pyraclostrobin, kresoxim-methyl, trifloxystrobin, famoxadone, fluoxastrobin, fenamidone, fluazinam, fentin hydroxide, cyprodinil, pyrimethanil, streptomycin, tetracycline, oxytetracycline, quinoxyfen, fludiosonil, iprodione, vinclozolin, chloroneb, dicloran, quintozene, etridiazole, propamocarb, dimethomorph, mandipropamid, triforine, fenarimol, imazalil, triflumizole, difenoconazole, fenbuconazole, ipconazole, metconazole, myclobutanil, propiconazole, prothioconazole, tebuconazole, tetraconazole, triadimefon, tridimenol, piperalin, fenhexamid, polyoxin, acibenzolar-s-methyl, cymoxanil, fosetyl-Al, phosphorous acid and salts, mineral oils, organic oils, potassium compounds, bicarbonates, copper, copper salts, sulfur, ferbam, mancozeb, maneb, metiram, thiram, ziram, captan, folpet, chlrothalonil, dodine, alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC, xylylcarb, acephate, azamethiphos, azinphos-ethyl, azinphos-methyl, cadusafos, chlorethoxyfos, chlorfenvinphos, chlormephos, chlorpyrifos, chlorpyrifos-methyl, coumaphos, cyanophos, demeton-s-methyl, diazinon, dichlorvos/ DDVP, dicrotophos, dimethoate, dimethylvinphos, disulfoton, EPN, ethion, ethoprophos, famphur, fenamiphos, fenitrothion, fenthion, fosthiazate, heptenophos, imicyfos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos- methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion, chlordane, endosulfan, ethiprole, fipronil, acrinathrin, allethrin, d-cis-trans allethrin, d-trans allethrin, bifenthrin, bioallethrin, bioallethrin s-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, permethrin, phenothrin, prallethrin, pyrethrin, resmethrin, RU 15525, silafluofen, tefluthrin, tetramethrin, tetramethrin, tralomethrin, transfluthrin, ZXI 8901, DDT, methoxychlor, acetamiprid, clothianidin, dinotefuran, imidacloprid, nitenpyram, thiacloprid, thiamethoxam, nicotine, spinetoram, spinosad, abamectin, emamectin benzoate, milbemectin, hydroprene, kinoprene, methoprene, fenoxycarb, pyriproxyfen, methyl bromide, methyl iodide, chloropicrin, sulfuryl fluoride, borax, and tartar emetic, pymetrozine, flonicamind, clofentexine, hexythiazox, etoxazole, Bacillus thuringiensis, Bacillus sphaericus, Bacillus thuringiensis subsp. israelensis, Bacillus sphaericus, Bacillus thuringiensis subsp. aizawai, Bacillus thuringiensis subsp. kurstaki, Bacillus thuringiensis subsp. tenebrionis, Cry1Ab, Cry1Ac, Cry1Fa, Cry2Ab, mCry3A, Cry3Ab, Cry3Bb, Cry34/35Ab1, diafenthiuron, azocyclotin, cyhexatin, fenbutatin oxide, propargite, tetradifon, chlorfenapyr, DNOC, bensultap, cartap hydrochloride, thiocyclam, thiosultap-sodium, bistrifluron, chlorfluazuron, diflubenzuron, flucycloxuron, flufenoxuron, hexaflumuron, lufenuron, novaluron, noviflumuron, feflubenzuron, triflumuron, buprofezin, cyromazine, chromafenozide, halofenozide, methoxyfenozide, tebufenozide, amitraz, fenazaquin, fenpyroximate, pyrimidifen, pyridaben, tebufenpyrad, tolfenpyrad, rotenone, cyenopyrafen, hydramethylnon, acequinocyl, fluacrypyrim, phosphine, aluminum phosphide, calcium phosphide, phosphine, zinc phosphide, cyanide, indoxacarb, metaflumizone, spirodiclofen, spiromesifen, spirotetramat, chlorantraniliprole, cyantraniliprole, flubendiamide, azadirachtin, benzoximate, bifenazate, chinomethionat, cryolite, dicofol, pyridalyl, cyflumetafen, ancymidol, butralin, alcohols, chlormequat chloride, cytokinins, daminozide, ethephon, ethylene, gibberellic acid and/or gibberellin mixtures, indole-3-butyric acid (IBA), maleic hydrazide and/or other potassium salts, mefluidide, mepiquat chloride and/or mepiquat pentaborate, naphthalene-acetic acid, 1-naphthaleneacetamide, n-decanol, paclobutrazol, prohexadione calcium, trinexapac-ethyl, uniconazole, fenoxasulfone, penthiopyrad, picoxystrobin, pyraoxystrobin, pyrametostrobin, penflufen and any combination thereof.

4. A method of increasing carotenoid levels in a plant comprising applying a composition comprising one or more herbicides, defoliants/desiccants, insecticides, nematicides, miticides, fungicides, antibiotics, plant growth regulators, or any combination thereof to a plant, wherein said one or more herbicides, defoliants/desiccants, insecticides, nematicides, miticides, fungicides, antibiotics, plant growth regulators are applied alone or in combination with a safener other pesticide or agrochemical in an amount sufficient to increase carotenoid levels in said plant.

5. The method according to claim 4, wherein said one or more herbicides, defoliants/desiccants, insecticides, nematicides, miticides, fungicides, antibiotics, plant growth regulators, has inhibit, activate, modulate, uncouple, up-regulate, mimic, disrupt, or otherwise modify: protoporphoryinogen IX oxidase (Protox), carotenoid biosynthesis, phytoene desaturase, deoxy-D-xylulose-5-phosphate reductoisomerase (DOXP reductoisomerase), p-hydroxyphenylpyruvate dioxygenase (HPPD), zeta-carotene desaturase (ZDS), 1-deoxy-D-xylulose-5-phosphate synthatase (DOXP synthase), lycopene cyclase (LC), and phytoene desaturase (PDS), Photosystem I in photosynthesis, Photosystem II in photosynthesis acetolactate synthase, 5-enolpyruvyl shikimate-3-phosphate synthase, glutamine synthetase microtubule organization or assembly in mitosis, formation of very long chain fatty acids, cellulose biosynthesis, auxin transport, internal auxins, nucleic acid synthesis; mitosis and/or cellular division; cellular respiration; amino acid and/or protein synthesis; lipids and/or membrane synthesis, cellular signal transduction, sterol biosynthesis, glucan synthesis, host-pathogen interactions, acetylcholinesterase, GABA-gated chloride channel antagonists, sodium channel modulators, nicotinic acetylcholine receptor agonists, nicotinic acetylcholine receptor allosteric activators, chloride channel activators, mimic juvenile hormones, Homopteran feeding, mite growth, activities of insect midgut membranes, mitochondrial ATP synthase, oxidative phosphorylation via proton gradient, nicotinic acetylcholine receptor channel blockers, chitin biosynthesis (type 1), molting disruptors (Dipteran), ecdysone receptor agonists, octopamine receptor agonists, mitochondrial complex III electron transport and mitochondrial complex I electron transport, voltage-dependent sodium channels, acetyl CoA carboxylase, mitochondrial complex IV electron transport and mitochondrial complex II electron transport, ryanodine receptors, gibberellic acid production, ethylene production, shoot elongation, cell division and cell elongation, ripening, overall plant growth and development.

6. The method according to claim 4, wherein said herbicides, defoliants/desiccants, insecticides, nematicides, miticides, fungicides, antibiotics, plant growth regulators, is selected from a group consisting of ametryn, bromacil, desmedipham, phenmedipham, hexazinone, atrazine, pyrazon, simazine, metribuzin, prometryn, prometon, amicarbazone, terbacil, bentazon, bromoxynil, ioxynil, pyridate, propanil, diuron, linuron, siduron, fluometuron, tebuthiuron, paraquat, diquat, sorgoleone, fischerellin A, juglone, anthroquinone, capsaicin, imazethapyr, imazapyr, imazaquin, imazamox imazamethabenz, imazapic, byspyribac, pyrithiobac, flucarbazone, thiencarbazone, propoxycarbazone, amidosulfuron, azimsulfuron, bensulfuron, chlorimuron, chlorsulfuron, ethoxysulfuron, flazasulfuron, flupyrsulfuron, foramsulfuron, halosulfuron, imazosulfuron, iodosulfuron, mesosulfuron, nicosulfuron, primisulfuron, rimsulfuron, sulfosulfuron, sulfometuron, thifensulfuron, tribenuron, trifloxysulfuron, triflusulfuron, cloransulam, florasulam, flumetsuam, penoxsulam, pyroxsulam, diclosulam, glyphosate, gliotoxin, bialaphos, fosmidomycin, mesotrione, sulcotrione, tembotrione, isoxaflutole, bicyclopyrone, pyrasulfatole, isoxachiortole, topramezone, benzofenap, pyrazoxyfen, DFPC, clomazone, norflurazon, flurochloridone, diflufenican, fluridone, picolinafen, flurtamone, beflubutamid, amitrole, CPTA acifluorfen, lactofen, oxyfluorfen, fomesafen, carfentrazone, sulfentrazone, azafenidin oxadiazon, oxadiargyl, cinidon, flumioxazin, flumiclorac, pyraflufen, fluazolate, saflufenacil, butafenacil, fluthiacet, leptospermone, pendimethalin, trifluralin, oryzalin, prodiamine, benefin, ethalfluralin, DCPA, butamiphos, amiprophos, pronamide, tebutam, carbetamide, propham, chlorpropham, clethodim, sethoxydim, tralkoxydim, fluazifop, fenoxaprop, quizalofop, diclofop, haloxyfop, pinoxaden, EPTC, pebulate, vernolate, molinate, triallate, butylate, bensulide, ethofumesate, benfuresate, TCA, dalapon, flupropanate, metazachlor, metolachlor, acetochlor, dimethenamid, pretilachlor, propachlor, alachlor, diphenamid, napropamide, naproanilide, flufenacet, mefenacet, fentrazamide, anilofos, piperophos, cafenstrole, indanofan, pyroxasulfone, dichlobenil, chlorthiamid, isoxaben, flupoxam, indaziflam, diflufenzopyr, naptalam, picloram, fluroxypyr, clopyralid, aminopyralid, triclopyr, aminocyclopyrachlor, 2,4-D, 2,4-DB, 2,4,5-T, dichlorprop, mecoprop, MCPA, MCPB, MCPP, dicamba, tricamba, chloramben, quinclorac, quinmerac, ailanthone, catechin, dimethipin, endothall, pelargonic acid, sodium chlorate, thiadiazuron, tribufos metalaxyl, mefenoxam, octhilinone, carbendazim, thisbendazole, zoxamide, flutolanil, carboxin, oxycarboxin, boscalid, azoxystrobin, pyraclostrobin, kresoxim-methyl, trifloxystrobin, famoxadone, fluoxastrobin, fenamidone, fluazinam, fentin hydroxide, cyprodinil, pyrimethanil, streptomycin, tetracycline, oxytetracycline, quinoxyfen, fludiosonil, iprodione, vinclozolin, chloroneb, dicloran, quintozene, etridiazole, propamocarb, dimethomorph, mandipropamid, triforine, fenarimol, imazalil, triflumizole, difenoconazole, fenbuconazole, ipconazole, metconazole, myclobutanil, propiconazole, prothioconazole, tebuconazole, tetraconazole, triadimefon, tridimenol, piperalin, fenhexamid, polyoxin, acibenzolar-s-methyl, cymoxanil, fosetyl-Al, phosphorous acid and salts, mineral oils, organic oils, potassium compounds, bicarbonates, copper, copper salts, sulfur, ferbam, mancozeb, maneb, metiram, thiram, ziram, captan, folpet, chlrothalonil, dodine, alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC, xylylcarb, acephate, azamethiphos, azinphos-ethyl, azinphos-methyl, cadusafos, chlorethoxyfos, chlorfenvinphos, chlormephos, chlorpyrifos, chlorpyrifos-methyl, coumaphos, cyanophos, demeton-s-methyl, diazinon, dichlorvos/ DDVP, dicrotophos, dimethoate, dimethylvinphos, disulfoton, EPN, ethion, ethoprophos, famphur, fenamiphos, fenitrothion, fenthion, fosthiazate, heptenophos, imicyfos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos- methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion, chlordane, endosulfan, ethiprole, fipronil, acrinathrin, allethrin, d-cis-trans allethrin, d-trans allethrin, bifenthrin, bioallethrin, bioallethrin s-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, permethrin, phenothrin, prallethrin, pyrethrin, resmethrin, RU 15525, silafluofen, tefluthrin, tetramethrin, tetramethrin, tralomethrin, transfluthrin, ZXI 8901, DDT, methoxychlor, acetamiprid, clothianidin, dinotefuran, imidacloprid, nitenpyram, thiacloprid, thiamethoxam, nicotine, spinetoram, spinosad, abamectin, emameetin benzoate, milbemectin, hydroprene, kinoprene, methoprene, fenoxycarb, pyriproxyfen, methyl bromide, methyl iodide, chloropicrin, sulfuryl fluoride, borax, and tartar emetic, pymetrozine, flonicamind, clofentexine, hexythiazox, etoxazole, Bacillus thuringiensis, Bacillus sphaericus, Bacillus thuringiensis subsp. israelensis, Bacillus sphaericus, Bacillus thuringiensis subsp. aizawai, Bacillus thuringiensis subsp. kurstaki, Bacillus thuringiensis subsp. tenebrionis, Cry1Ab, Cry1Ac, Cry1Fa, Cry2Ab, mCry3A, Cry3Ab, Cry3Bb, Cry34/35Ab1, diafenthiuron, azocyclotin, cyhexatin, fenbutatin oxide, propargite, tetradifon, chlorfenapyr, DNOC, bensultap, cartap hydrochloride, thiocyclam, thiosultap-sodium, bistrifluron, chlorfluazuron, diflubenzuron, flucycloxuron, flufenoxuron, hexaflumuron, lufenuron, novaluron, noviflumuron, feflubenzuron, triflumuron, buprofezin, cyromazine, chromafenozide, halofenozide, methoxyfenozide, tebufenozide, amitraz, fenazaquin, fenpyroximate, pyrimidifen, pyridaben, tebufenpyrad, tolfenpyrad, rotenone, cyenopyrafen, hydramethylnon, acequinocyl, fluacrypyrim, phosphine, aluminum phosphide, calcium phosphide, phosphine, zinc phosphide, cyanide, indoxacarb, metaflumizone, spirodiclofen, spiromesifen, spirotetramat, chlorantraniliprole, cyantraniliprole, flubendiamide, azadirachtin, benzoximate, bifenazate, chinomethionat, cryolite, dicofol, pyridalyl, cyflumetafen, ancymidol, butralin, alcohols, chlormequat chloride, cytokinins, daminozide, ethephon, ethylene, gibberellic acid and/or gibberellin mixtures, indole-3-butyric acid (IBA), maleic hydrazide and/or other potassium salts, mefluidide, mepiquat chloride and/or mepiquat pentaborate, naphthalene-acetic acid, 1-naphthaleneacetamide, n-decanol, paclobutrazol, prohexadione calcium, trinexapac-ethyl, uniconazole, fenoxasulfone, penthiopyrad, picoxystrobin, pyraoxystrobin, pyrametostrobin, penflufen and any combination thereof

7. The method according to claim 1, wherein said carbohydrate is selected from cellulose, sugars, starches or fiber.

8. The method according to claim 7, wherein said carbohydrate is selected from sucrose, fructose, glucose, maltose, or raffanose.

9. The method according to claim 7, wherein said fiber is either acid detergent fiber or neutral detergent fiber.

10. The method according to claim 4, wherein the carotenoid is beta-carotene, lycopene, lutein, zeaxanthin, violaxanthin, neoxanthin, or antheraxanthin.

11. A method of increasing amino acids and/or protein levels in a plant comprising applying a composition comprising one or more herbicides, defoliants/desiccants, insecticides, nematicides, miticides, fungicides, antibiotics, plant growth regulators, or any combination thereof to a plant, wherein said one or more herbicides, defoliants/desiccants, insecticides, nematicides, miticides, fungicides, antibiotics, plant growth regulators are applied alone or in combination with a safener other pesticide or agrochemical in an amount sufficient to increase amino acids and/or protein levels in said plant.

12. The method according to claim 11, wherein said one or more herbicides, defoliants/desiccants, insecticides, nematicides, miticides, fungicides, antibiotics, plant growth regulators, has inhibit, activate, modulate, uncouple, up-regulate, mimic, disrupt, or otherwise modify: protoporphoryinogen IX oxidase (Protox), carotenoid biosynthesis, phytoene desaturase, deoxy-D-xylulose-5-phosphate reductoisomerase (DOXP reductoisomerase), p-hydroxyphenylpyruvate dioxygenase (HPPD), zeta-carotene desaturase (ZDS), 1-deoxy-D-xylulose-5-phosphate synthatase (DOXP synthase), lycopene cyclase (LC), and phytoene desaturase (PDS), Photosystem I in photosynthesis, Photosystem II in photosynthesis acetolactate synthase, 5-enolpyruvylshikimate-3-phosphate synthase, glutamine synthetase microtubule organization or assembly in mitosis, formation of very long chain fatty acids, cellulose biosynthesis, auxin transport, internal auxins, nucleic acid synthesis; mitosis and/or cellular division; cellular respiration; amino acid and/or protein synthesis; lipids and/or membrane synthesis, cellular signal transduction, sterol biosynthesis, glucan synthesis, host-pathogen interactions, acetylcholinesterase, GABA-gated chloride channel antagonists, sodium channel modulators, nicotinic acetylcholine receptor agonists, nicotinic acetylcholine receptor allosteric activators, chloride channel activators, mimic juvenile hormones, Homopteran feeding, mite growth, activities of insect midgut membranes, mitochondrial ATP synthase, oxidative phosphorylation via proton gradient, nicotinic acetylcholine receptor channel blockers, chitin biosynthesis (type 1), molting disruptors (Dipteran), ecdysone receptor agonists, octopamine receptor agonists, mitochondrial complex III electron transport and mitochondrial complex I electron transport, voltage-dependent sodium channels, acetyl CoA carboxylase, mitochondrial complex IV electron transport and mitochondrial complex II electron transport, ryanodine receptors, gibberellic acid production, ethylene production, shoot elongation, cell division and cell elongation, ripening, overall plant growth and development.

13. The method according to claim 11, wherein said herbicides, defoliants/desiccants, insecticides, nematicides, miticides, fungicides, antibiotics, plant growth regulators, is selected from a group consisting of ametryn, bromacil, desmedipham, phenmedipham, hexazinone, atrazine, pyrazon, simazine, metribuzin, prometryn, prometon, amicarbazone, terbacil, bentazon, bromoxynil, ioxynil, pyridate, propanil, diuron, linuron, siduron, fluometuron, tebuthiuron, paraquat, diquat, sorgoleone, fischerellin A, juglone, anthroquinone, capsaicin, imazethapyr, imazapyr, imazaquin, imazamox imazamethabenz, imazapic, byspyribac, pyrithiobac, flucarbazone, thiencarbazone, propoxycarbazone, amidosulfuron, azimsulfuron, bensulfuron, chlorimuron, chlorsulfuron, ethoxysulfuron, flazasulfuron, flupyrsulfuron, foramsulfuron, halosulfuron, imazosulfuron, iodosulfuron, mesosulfuron, nicosulfuron, primisulfuron, rimsulfuron, sulfosulfuron, sulfometuron, thifensulfuron, tribenuron, trifloxysulfuron, triflusulfuron, cloransul am, florasulam, flumetsuam, penoxsulam, pyroxsulam, diclosulam, glyphosate, gliotoxin, bialaphos, fosmidomycin, mesotrione, sulcotrione, tembotrione, isoxaflutole, bicyclopyrone, pyrasulfatole, isoxachlortole, topramezone, benzofenap, pyrazoxyfen, DFPC, clomazone, norflurazon, flurochloridone, diflufenican, fluridone, picolinafen, flurtamone, beflubutamid, amitrole, CPTA acifluorfen, lactofen, oxyfluorfen, fomesafen, carfentrazone, sulfentrazone, azafenidin oxadiazon, oxadiargyl, cinidon, flumioxazin, flumiclorac, pyraflufen, fluazolate, saflufenacil, butafenacil, fluthiacet, leptospermone, pendimethalin, trifluralin, oryzalin, prodiamine, benefin, ethalfluralin, DCPA, butamiphos, amiprophos, pronamide, tebutam, carbetamide, propham, chlorpropham, clethodim, sethoxydim, tralkoxydim, fluazifop, fenoxaprop, quizalofop, diclofop, haloxyfop, pinoxaden, EPTC, pebulate, vernolate, molinate, triallate, butylate, bensulide, ethofumesate, benfuresate, TCA, dalapon, flupropanate, metazachlor, metolachlor, acetochlor, dimethenamid, pretilachlor, propachlor, alachlor, diphenamid, napropamide, naproanilide, flufenacet, mefenacet, fentrazamide, anilofos, piperophos, cafenstrole, indanofan, pyroxasulfone, dichlobenil, chlorthiamid, isoxaben, flupoxam, indaziflam, diflufenzopyr, naptalam, picloram, fluroxypyr, clopyralid, aminopyralid, triclopyr, aminocyclopyrachlor, 2,4-D, 2,4-DB, 2,4,5-T, dichlorprop, mecoprop, MCPA, MCPB, MCPP, dicamba, tricamba, chloramben, quinclorac, quinmerac, ailanthone, catechin, dimethipin, endothall, pelargonic acid, sodium chlorate, thiadiazuron, tribufos metalaxyl, mefenoxam, octhilinone, carbendazim, thisbendazole, zoxamide, flutolanil, carboxin, oxycarboxin, boscalid, azoxystrobin, pyraclostrobin, kresoxim-methyl, trifloxystrobin, famoxadone, fluoxastrobin, fenamidone, fluazinam, fentin hydroxide, cyprodinil, pyrimethanil, streptomycin, tetracycline, oxytetracycline, quinoxyfen, fludiosonil, iprodione, vinclozolin, chloroneb, dicloran, quintozene, etridiazole, propamocarb, dimethomorph, mandipropamid, triforine, fenarimol, imazalil, triflumizole, difenoconazole, fenbuconazole, ipconazole, metconazole, myclobutanil, propiconazole, prothioconazole, tebuconazole, tetraconazole, triadimefon, tridimenol, piperalin, fenhexamid, polyoxin, acibenzolar-s-methyl, cymoxanil, fosetyl-Al, phosphorous acid and salts, mineral oils, organic oils, potassium compounds, bicarbonates, copper, copper salts, sulfur, ferbam, mancozeb, maneb, metiram, thiram, ziram, captan, folpet, chlrothalonil, dodine, alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC, xylylcarb, acephate, azamethiphos, azinphos-ethyl, azinphos-methyl, cadusafos, chlorethoxyfos, chlorfenvinphos, chlormephos, chlorpyrifos, chlorpyrifos-methyl, coumaphos, cyanophos, demeton-s-methyl, diazinon, dichlorvos/ DDVP, dicrotophos, dimethoate, dimethylvinphos, disulfoton, EPN, ethion, ethoprophos, famphur, fenamiphos, fenitrothion, fenthion, fosthiazate, heptenophos, imicyfos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos- methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion, chlordane, endosulfan, ethiprole, fipronil, acrinathrin, allethrin, d-cis-trans allethrin, d-trans allethrin, bifenthrin, bioallethrin, bioallethrin s-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, permethrin, phenothrin, prallethrin, pyrethrin, resmethrin, RU 15525, silafluofen, tefluthrin, tetramethrin, tetramethrin, tralomethrin, transfluthrin, ZXI 8901, DDT, methoxychlor, acetamiprid, clothianidin, dinotefuran, imidacloprid, nitenpyram, thiacloprid, thiamethoxam, nicotine, spinetoram, spinosad, abamectin, emamectin benzoate, milbemectin, hydroprene, kinoprene, methoprene, fenoxycarb, pyriproxyfen, methyl bromide, methyl iodide, chloropicrin, sulfuryl fluoride, borax, and tartar emetic, pymetrozine, flonicamind, clofentexine, hexythiazox, etoxazole, Bacillus thuringiensis, Bacillus sphaericus, Bacillus thuringiensis subsp. israelensis, Bacillus sphaericus, Bacillus thuringiensis subsp. aizawai, Bacillus thuringiensis subsp. kurstaki, Bacillus thuringiensis subsp. tenebrionis, Cry1Ab, Cry1Ac, Cry1Fa, Cry2Ab, mCry3A, Cry3Ab, Cry3Bb, Cry34/35Ab1, diafenthiuron, azocyclotin, cyhexatin, fenbutatin oxide, propargite, tetradifon, chlorfenapyr, DNOC, bensultap, cartap hydrochloride, thiocyclam, thiosultap-sodium, bistrifluron, chlorfluazuron, diflubenzuron, flucycloxuron, flufenoxuron, hexaflumuron, lufenuron, novaluron, noviflumuron, feflubenzuron, triflumuron, buprofezin, cyromazine, chromafenozide, halofenozide, methoxyfenozide, tebufenozide, amitraz, fenazaquin, fenpyroximate, pyrimidifen, pyridaben, tebufenpyrad, tolfenpyrad, rotenone, cyenopyrafen, hydramethylnon, acequinocyl, fluacrypyrim, phosphine, aluminum phosphide, calcium phosphide, phosphine, zinc phosphide, cyanide, indoxacarb, metaflumizone, spirodiclofen, spiromesifen, spirotetramat, chlorantraniliprole, cyantraniliprole, flubendiamide, azadirachtin, benzoximate, bifenazate, chinomethionat, cryolite, dicofol, pyridalyl, cyflumetafen, ancymidol, butralin, alcohols, chlormequat chloride, cytokinins, daminozide, ethephon, ethylene, gibberellic acid and/or gibberellin mixtures, indole-3-butyric acid (IBA), maleic hydrazide and/or other potassium salts, mefluidide, mepiquat chloride and/or mepiquat pentaborate, naphthalene-acetic acid, 1-naphthaleneacetamide, n-decanol, paclobutrazol, prohexadione calcium, trinexapac-ethyl, uniconazole, fenoxasulfone, penthiopyrad, picoxystrobin, pyraoxystrobin, pyrametostrobin, penflufen and any combination thereof.

14. The method according to claim 11, wherein the amino acid is selected from the group consisting of glutamine, leucine, arginine, histidine, isoleucine, lysine, methionine, phenylalanine, threonine, tryptophan, tyrosine, valine, alanine, asparagine, aspartate, cysteine, glutamate, glycine, proline, serine, glutamic acid, and any combination thereof

15. A method of increasing levels of minerals in a plant comprising applying a composition comprising one or more herbicides, defoliants/desiccants, insecticides, nematicides, miticides, fungicides, antibiotics, plant growth regulators, or any combination thereof wherein said one or more herbicides, defoliants/desiccants, insecticides, nematicides, miticides, fungicides, antibiotics, and/or plant growth regulators are applied alone or in combination with a safener, other herbicides, defoliants/desiccants, insecticides, nematicides, miticides, fungicides, antibiotics, and/or plant growth regulators or certain other pesticides/agrochemical in an amount sufficient to increase levels of minerals in said plant.

16. The method according to claim 15, wherein said one or more herbicides, defoliants/desiccants, insecticides, nematicides, miticides, fungicides, antibiotics, plant growth regulators, has inhibit, activate, modulate, uncouple, up-regulate, mimic, disrupt, or otherwise modify: protoporphoryinogen IX oxidase (Protox), carotenoid biosynthesis, phytoene desaturase, deoxy-D-xylulose-5-phosphate reductoisomerase (DOXP reductoisomerase), p-hydroxyphenylpyruvate dioxygenase (HPPD), zeta-carotene desaturase (ZDS), 1-deoxy-D-xylulose-5-phosphate synthatase (DOXP synthase), lycopene cyclase (LC), and phytoene desaturase (PDS), Photosystem I in photosynthesis, Photosystem II in photosynthesis acetolactate synthase, 5-enolpyruvylshikimate-3-phosphate synthase, glutamine synthetase microtubule organization or assembly in mitosis, formation of very long chain fatty acids, cellulose biosynthesis, auxin transport, internal auxins, nucleic acid synthesis; mitosis and/or cellular division; cellular respiration; amino acid and/or protein synthesis; lipids and/or membrane synthesis, cellular signal transduction, sterol biosynthesis, glucan synthesis, host-pathogen interactions, acetylcholinesterase, GABA-gated chloride channel antagonists, sodium channel modulators, nicotinic acetylcholine receptor agonists, nicotinic acetylcholine receptor allosteric activators, chloride channel activators, mimic juvenile hormones, Homopteran feeding, mite growth, activities of insect midgut membranes, mitochondrial ATP synthase, oxidative phosphorylation via proton gradient, nicotinic acetylcholine receptor channel blockers, chitin biosynthesis (type 1), molting disruptors (Dipteran), ecdysone receptor agonists, octopamine receptor agonists, mitochondria] complex III electron transport and mitochondrial complex I electron transport, voltage-dependent sodium channels, acetyl CoA carboxylase, mitochondrial complex IV electron transport and mitochondrial complex II electron transport, ryanodine receptors, gibberellic acid production, ethylene production, shoot elongation, cell division and cell elongation, ripening, overall plant growth and development.

17. The method according to claim 15, wherein said herbicides, defoliants/desiccants, insecticides, nematicides, miticides, fungicides, antibiotics, plant growth regulators, are from a group consisting of ametryn, bromacil, desmedipham, phenmedipham, hexazinone, atrazine, pyrazon, simazine, metribuzin, prometryn, prometon, amicarbazone, terbacil, bentazon, bromoxynil, ioxynil, pyridate, propanil, diuron, linuron, siduron, fluometuron, tebuthiuron, paraquat, diquat, sorgoleone, fischerellin A, juglone, anthroquinone, capsaicin, imazethapyr, imazapyr, imazaquin, imazamox imazamethabenz, imazapic, byspyribac, pyrithiobac, flucarbazone, thiencarbazone, propoxycarbazone, amidosulfuron, azimsulfuron, bensulfuron, chlorimuron, chlorsulfuron, ethoxysulfuron, flazasulfuron, flupyrsulfuron, foramsulfuron, halosulfuron, imazosulfuron, iodosulfuron, mesosulfuron, nicosulfuron, primisulfuron, rimsulfuron, sulfosulfuron, sulfometuron, thifensulfuron, tribenuron, trifloxysulfuron, triflusulfuron, cloransulam, florasulam, flumetsuam, penoxsulam, pyroxsulam, diclosulam, glyphosate, gliotoxin, bialaphos, fosmidomycin, mesotrione, sulcotrione, tembotrione, isoxaflutole, bicyclopyrone, pyrasulfatole, isoxachlortole, topramezone, benzofenap, pyrazoxyfen, DFPC, clomazone, norflurazon, flurochloridone, diflufenican, fluridone, picolinafen, flurtamone, beflubutamid, amitrole, CPTA acifluorfen, lactofen, oxyfluorfen, fomesafen, carfentrazone, sulfentrazone, azafenidin oxadiazon, oxadiargyl, cinidon, flumioxazin, flumiclorac, pyraflufen, fluazolate, saflufenacil, butafenacil, fluthiacet, leptospermone, pendimethalin, trifluralin, oryzalin, prodiamine, benefin, ethaffluralin, DCPA, butamiphos, amiprophos, pronamide, tebutam, carbetamide, propham, chlorpropham, clethodim, sethoxydim, tralkoxydim, fluazifop, fenoxaprop, quizalofop, diclofop, haloxyfop, pinoxaden, EPTC, pebulate, vernolate, molinate, triallate, butylate, bensulide, ethofumesate, benfuresate, TCA, dalapon, flupropanate, metazachlor, metolachlor, acetochlor, dimethenamid, pretilachlor, propachlor, alachlor, diphenamid, napropamide, naproanilide, flufenacet, mefenacet, fentrazamide, anilofos, piperophos, cafenstrole, indanofan, pyroxasulfone, dichlobenil, chlorthiamid, isoxaben, flupoxam, indaziflam, diflufenzopyr, naptalam, picloram, fluroxypyr, clopyralid, aminopyralid, triclopyr, aminocyclopyrachlor, 2,4-D, 2,4-DB, 2,4,5-T, dichlorprop, mecoprop, MCPA, MCPB, MCPP, dicamba, tricamba, chloramben, quinclorac, quinmerac, ailanthone, catechin, dimethipin, endothall, pelargonic acid, sodium chlorate, thiadiazuron, tribufos metalaxyl, mefenoxam, octhilinone, carbendazim, thisbendazole, zoxamide, flutolanil, carboxin, oxycarboxin, boscalid, azoxystrobin, pyraclostrobin, kresoxim-methyl, trifloxystrobin, famoxadone, fluoxastrobin, fenamidone, fluazinam, fentin hydroxide, cyprodinil, pyrimethanil, streptomycin, tetracycline, oxytetracycline, quinoxyfen, fludiosonil, iprodione, vinelozolin, chloroneb, dicloran, quintozene, etridiazole, propamocarb, dimethomorph, mandipropamid, triforine, fenarimol, imazalil, triflumizole, difenoconazole, fenbuconazole, ipconazole, metconazole, myclobutanil, propiconazole, prothioconazole, tebuconazole, tetraconazole, triadimefon, tridimenol, piperalin, fenhexamid, polyoxin, acibenzolar-s-methyl, cymoxanil, fosetyl-Al, phosphorous acid and salts, mineral oils, organic oils, potassium compounds, bicarbonates, copper, copper salts, sulfur, ferbam, mancozeb, maneb, metiram, thiram, ziram, captan, folpet, chlrothalonil, dodine, alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC, xylylcarb, acephate, azamethiphos, azinphos-ethyl, azinphos-methyl, cadusafos, chlorethoxyfos, chlorfenvinphos, chlormephos, chlorpyrifos, chlorpyrifos-methyl, coumaphos, cyanophos, demeton-s-methyl, diazinon, dichlorvos/ DDVP, dicrotophos, dimethoate, dimethylvinphos, disulfoton, EPN, ethion, ethoprophos, famphur, fenamiphos, fenitrothion, fenthion, fosthiazate, heptenophos, imicyfos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos- methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion, chlordane, endosulfan, ethiprole, fipronil, acrinathrin, allethrin, d-cis-trans allethrin, d-trans allethrin, bifenthrin, bioallethrin, bioallethrin s-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, permethrin, phenothrin, prallethrin, pyrethrin, resmethrin, RU 15525, silafluofen, tefluthrin, tetramethrin, tetramethrin, tralomethrin, transfluthrin, ZXI 8901, DDT, methoxychlor, acetamiprid, clothianidin, dinotefuran, imidacloprid, nitenpyram, thiacloprid, thiamethoxam, nicotine, spinetoram, spinosad, abamectin, emamectin benzoate, milbemectin, hydroprene, kinoprene, methoprene, fenoxycarb, pyriproxyfen, methyl bromide, methyl iodide, chloropicrin, sulfuryl fluoride, borax, and tartar emetic, pymetrozine, flonicamind, clofentexine, hexythiazox, etoxazole, Bacillus thuringiensis, Bacillus sphaericus, Bacillus thuringiensis subsp. israelensis, Bacillus sphaericus, Bacillus thuringiensis subsp. aizawai, Bacillus thuringiensis subsp. kurstaki, Bacillus thuringiensis subsp. tenebrionis, Cry1Ab, Cry1Ac, Cry1Fa, Cry2Ab, mCry3A, Cry3Ab, Cry3Bb, Cry34/35Ab1, diafenthiuron, azocyclotin, cyhexatin, fenbutatin oxide, propargite, tetradifon, chlorfenapyr, DNOC, bensultap, cartap hydrochloride, thiocyclam, thiosultap-sodium, bistrifluron, chlorfluazuron, diflubenzuron, flucycloxuron, flufenoxuron, hexaflumuron, lufenuron, novaluron, noviflumuron, feflubenzuron, triflumuron, buprofezin, cyromazine, chromafenozide, halofenozide, methoxyfenozide, tebufenozide, amitraz, fenazaquin, fenpyroximate, pyrimidifen, pyridaben, tebufenpyrad, tolfenpyrad, rotenone, cyenopyrafen, hydramethylnon, acequinocyl, fluacrypyrim, phosphine, aluminum phosphide, calcium phosphide, phosphine, zinc phosphide, cyanide, indoxacarb, metaflumizone, spirodiclofen, spiromesifen, spirotetramat, chlorantraniliprole, cyantraniliprole, flubendiamide, azadirachtin, benzoximate, bifenazate, chinomethionat, cryolite, dicofol, pyridalyl, cyflumetafen, ancymidol, butralin, alcohols, chlormequat chloride, cytokinins, daminozide, ethephon, ethylene, gibberellic acid and/or gibberellin mixtures, indole-3-butyric acid (IBA), maleic hydrazide and/or other potassium salts, mefluidide, mepiquat chloride and/or mepiquat pentaborate, naphthalene-acetic acid, 1-naphthaleneacetamide, n-decanol, paclobutrazol, prohexadione calcium, trinexapac-ethyl, uniconazole, fenoxasulfone, penthiopyrad, picoxystrobin, pyraoxystrobin, pyrametostrobin, penflufen and any combination thereof.

18. The method according to claim 17, wherein the minerals are selected from the group consisting of N, P, K, Ca, Mg, S, Cl, Co, Cu, Fe, Mn, Mo, Na, Ni, Zn, and any combination thereof.

19. A method of increasing levels of anti-oxidant compounds in a plant comprising applying a composition comprising one or more herbicides, defoliants/desiccants, insecticides, nematicides, miticides, fungicides, antibiotics, plant growth regulators, or any combination thereof; wherein said one or more herbicides, defoliants/desiccants, insecticides, nematicides, miticides, fungicides, antibiotics, and/or plant growth regulators are applied alone or in combination with a safener, other herbicides, defoliants/desiccants, insecticides, nematicides, miticides, fungicides, antibiotics, and/or plant growth regulators or certain other pesticides/agrochemical in an amount sufficient to increase levels of anti-oxidant compounds in said plant.

20. A method of improving tolerance to abiotic stress conditions of a plant comprising applying a composition comprising one or more herbicides, defoliants/desiccants, insecticides, nematicides, miticides, fungicides, antibiotics, plant growth regulators, or any combination thereof; wherein said one or more herbicides, defoliants/desiccants, insecticides, nematicides, miticides, fungicides, antibiotics, and/or plant growth regulators are applied alone or in combination with a safener, other herbicides, defoliants/desiccants, insecticides, nematicides, miticides, fungicides, antibiotics, and/or plant growth regulators or certain other pesticides/agrochemical in an amount sufficient to improve tolerance to abiotic stress conditions in said plant.

21. A method for identifying a composition that improves the production of a phytonutrient of interest, comprising:
a) applying a candidate composition to one or more plant;
b) growing said one or more plants treated with the candidate composition and harvesting at least part of the plant;
c) measuring or determining levels/concentrations/amounts of a phytonutrient of interest in the harvested part of said one or more treated plant;
d) comparing the levels of said phytonutrient of interest in said treated and the levels of said phytonutrient of interest in untreated plants; and
e) identifying the candidate composition as a composition that improves the production of the phytonutrient of interest, if the level of the phytonutrient in the harvested part of the treated plant is higher than that of the plant untreated with the candidate composition; wherein the candidate composition comprises a herbicide, a defoliant/desiccant, an insecticide, a nematicide, a miticide, a fungicide, an antibiotic, a plant growth regulator, or any combination thereof; and wherein the phytonutrient of interest is a lipid, a vitamin, a protein, an amino acid, a carbohydrate, a carotenoid, a glucosinolate, an antioxidant, a mineral, a phenolic, or any combination thereof

22. A method comprising applying a composition comprising one or more safener alone or in combination with herbicides, defoliants/desiccants, insecticides, nematicides, miticides, fungicides, antibiotics, plant growth regulators, or any combination thereof to a plant, wherein said one or more safener is applied alone or in combination with a safener other pesticide or agrochemical in an amount sufficient to increase:
a) carbohydrate levels in said plant;
b) carotenoid or antioxidant levels in a plant;
c) amino acids and/or protein levels in said plant; or
d) levels of minerals in said plant.

23. The method according to claim 22, wherein said safener is selected from benoxacor, cloquintocet, cyometrinil, cyprosulfamide, dichlormid, dicyclonon, dietholate, fenchlorazole, fenclorim, flurazole, fluxofenim, furilazole, isoxadifen, jiecaowan, jiecaoxi, mefenpyr, mephenate, naphthalic anhydride, oxabetrinil, daimuron, mefenpyr-diethy, cloquintocet-mexyl, isoxadifen-ethyl, fenchlorazole-ethyl or MG 191.

24. The method according to claim 22, wherein said carbohydrate is selected from cellulose, sugars, starches or fiber.

25. The method according to claim 24, wherein said carbohydrate is selected from sucrose, fructose, glucose, maltose, or raffanose.

26. The method according to claim 24, wherein said fiber is either acid detergent fiber or neutral detergent fiber.

27. The method according to claim 22, wherein the carotenoid is beta-carotene, lycopene, lutein, zeaxanthin, violaxanthin, neoxanthin, or antheraxanthin.

28. The method according to claim 22, wherein the amino acid is selected from the group consisting of glutamine, leucine, arginine, histidine, isoleucine, lysine, methionine, phenylalanine, threonine, tryptophan, tyrosine, valine, alanine, asparagine, aspartate, cysteine, glutamate, glycine, proline, serine, glutamic acid, and any combination thereof

29. The method according to claim 22, wherein the minerals are selected from the group consisting of N, P, K, Ca, Mg, S, Cl, Co, Cu, Fe, Mn, Mo, Na, Ni, Zn, and any combination thereof

30. The method according to claim 22, wherein the anti-oxidant compound is selected from the group consisting of carotenoids, essential oils, flavonoids, anthocyanins, alkaloids, glucosinolates, vitamins and any combination thereof.
